(19) United States
(12) Patent Application Publication
(10) Pub. No.: US 2023/0233443 A1
(43) Pub. Date: Jul. 27, 2023

(54) COSMETIC COMPOSITION COMPRISING A GRAFTED POLYHYDROXYALKANOATE COPOLYMER IN A FATTY MEDIUM

(71) Applicant: L’OREAL, Paris (FR)

(72) Inventors: Sandrine CHODOROWSKI-KIMMES, Aulnay-Sous-Bois (FR); Julien PORTAL, Aulnay-Sous-Bois (FR); Romain GARCON, Aulnay-Sous-Bois (FR); Etienne SAVONNET, Aulnay-Sous-Bois (FR)

(21) Appl. No.: 17/794,727

(22) PCT Filed: Jun. 23, 2021
(86) PCT No.: PCT/EP2021/067264

§ 371 Date: Jul. 22, 2022

(30) Foreign Application Priority Data
Jun. 24, 2020 (FR) 2006594

(51) Int. Cl. A61K 8/85; A61Q 19/00; A61K 8/31; C08G 63/06; C08G 63/91

(52) U.S. Cl. CPC A61K 8/85; A61Q 19/00; A61K 8/31; C08G 63/06; C08G 63/912; A61K 2800/85; A61K 2800/10

(57) ABSTRACT

The present invention relates to a cosmetic composition comprising a) one or more polyhydroxyalkanoate (PH A) copolymers which contain, and preferably consist of, at least two different repeating polymer units chosen from the units (A) and (B) below, and also the optical or geometrical isomers thereof, the organic or mineral acid or base salts thereof, and the solvates thereof such as hydrates: -[-0-CH(R¹)—CH₂—C(0)-]- unit (A) -[-0-CH(R²)—CH₂—C(0)-]- unit (B) in which polymer units (A) and (B): —R¹ represents a hydrocarbon-based chain chosen from i) linear or branched (C₅-C₂₈)alkyl, ii) linear or branched (C₆-C₂₈)alkenyl, iii) linear or branched (C₆-C₂₈)alkynyl; preferably, the hydrocarbon-based group is linear; said hydrocarbon-based chain being optionally substituted and/or interrupted with atoms or groups as described in the description; —R² represents a cyclic or non-cyclic, linear or branched, saturated or unsaturated hydrocarbon-based group, comprising from 3 to 30 carbon atoms; b) a fatty medium comprising one or more fatty substances which are preferably liquid at 25° C. and at atmospheric pressure; it being understood that (A) is different from (B).

The present invention relates to a cosmetic composition comprising a polyhydroxyalkanoate copolymer bearing grafted or functionalized hydrocarbon-based groups in a fatty medium, and also to a process for treating keratin materials using such a composition.

It is known practice to use, in cosmetics, film-forming polymers which can be conveyed in organic media, such as hydrocarbon-based oils. Polymers are notably used as film-forming agents in makeup products such as mascaras, eyeliners, eyeshadows or lipsticks.

FR-A-2964663 describes a cosmetic composition comprising pigments coated with a C₃-C₂₁ polyhydroxyalkanoate, such as poly(hydroxybutyrate-co-hydroxyvalerate).

WO 2011/154508 describes a cosmetic composition comprising a 4-carboxy-2-pyrrolidinone ester derivative and a film-forming polymer which may be a polyhydroxyalkanoate, such as polyhydroxybutyrate, polyhydroxyvalerate and polyhydroxybutyrate-co-polyhydroxyvalerate.

US-A-2015/274972 describes a cosmetic composition comprising a thermoplastic resin, such as a polyhydroxyalkanoate, in aqueous dispersion and a silicone elastomer. On the other hand, WO 2018/178899 describes a cosmetic composition comprising at least one polyhydroxyalkanoate (PHA) in the form of particles with an average diameter (d50) from 0.1 μm to 100 μm, in an amount of from 0.1% by weight to 30% by weight, with respect to the total weight of the composition.in order to absorb oily substances, such as sebum. However most PHAs are not solubilized satisfactorily in fatty substances such as volatile oil as isododecane.

The majority of the polyhydroxyalkanoates are polymers derived from the polycondensation of polymeric repeating units that are for the most part identical and derived from the same carbon source or substrate. These documents do not describe the use of copolymers derived from polycondensation using an aliphatic substrate or first carbon source, and at least one second substrate different from the first, comprising one or more reactive functions, the chemical nature of which is different from that of the first carbon source.

There is thus a need for a composition comprising PHAs with varied functionalization or which are functionalizable with lipophilic or non-lipophilic active agents, which can make them active and soluble in a fatty phase. This makes it possible to obtain a film on keratin materials which has good cosmetic properties, notably good resistance to oils and to sebum, and also to be able to modify the gloss or the mattness.

The Applicant has discovered that polyhydroxyalkanoate copolymers bearing particular grafted or functionalized hydrocarbon-based groups, as defined below, may be readily used in fatty media, thus making it possible to obtain homogeneous compositions. Moreover, the PHA according to the invention are film forming polymers. The composition shows good stability, notably after storage for one month at room temperature (25° C.). The composition, notably after its application to keratin materials, makes it possible to obtain a film having good cosmetic properties, in particular good resistance to oils and to sebum, and also a matt or glossy appearance. Furthermore, by grafting, it is possible to introduce into the polymer active agents, notably organic active agents, such as UV-screening agents, fluorescent or non-fluorescent chromophores, anti-ageing active agents, said active agents then being able to become more resistant once they have been grafted, notably resistant to oils, water and sebum.

Thus, the main subject of the present invention is a composition comprising:

a) one or more polyhydroxyalkanoate (PHA) copolymers which contain, and preferably consist of, at least two different repeating polymer units chosen from the units (A) and (B) below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

in which polymer units (A) and (B):
R¹ represents a hydrocarbon-based chain chosen from i) linear or branched (C₅-C₂₈)alkyl, ii) linear or branched (C₆-C₂₈)alkenyl, iii) linear or branched (C₆-C₂₈)alkynyl; preferably, the hydrocarbon-based group is linear;
said hydrocarbon-based chain being:
   substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl, naphtyl or furyl, and j) (hetero)cycloalkyl such as anhydride, or epoxide, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent as defined previously and X representing a′) O, S, N(Rₐ) or Si(R_{b})(R_{c}), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl, preferably Rₐ represents a hydrogen atom; R_{b} and R_{c}, which may be identical or different, represent a (C₁-C₄)alkyl or (C₁-C₄)alkoxy group, particularly only one substituent-; preferably chosen from b) halogen, and j) such as epoxide; and/or
   interrupted with one or more heteroatoms a′) such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide with r being equal to 1 or 2, Rₐ being as defined previously, preferably Rₐ represents a hydrogen atom, R_{b} and R_{c} being as defined previously;
R² represents a cyclic or non-cyclic, linear or branched, saturated or unsaturated hydrocarbon-based group comprising from 3 to 30 carbon atoms; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted; and
   b) a fatty medium comprising one or more fatty substances; which are preferably liquid at 25° C. and at atmospheric pressure;
   it being understood that (A) is different from (B).

Another subject of the invention is the use in cosmetics of a) one or more PHA copolymers as defined previously and b) one or more fatty substances as defined previously in cosmetics.

Another subject of the invention is a process for treating keratin materials, preferably α) keratin fibres, notably human keratin fibres such as the hair, or β) human skin, in particular the lips, using a) one or more PHA copolymers as defined previously and b) one or more fatty substances as defined previously.

Another subject of the invention is novel grafted PHA copolymers.

More particularly, a subject of the invention is a non-therapeutic cosmetic process for treating keratin materials, comprising the application to the keratin materials of a composition as defined previously. The treatment process is in particular a process for caring for or making up keratin materials.

For the purposes of the present invention and unless otherwise indicated:
the term “cosmetic active agent” means the radical of an organic or organosilicon compound which can be integrated into a cosmetic composition to give an effect on keratin materials, whether this effect is immediate or provided by repeated applications. As examples of cosmetic active agents, mention may be made of coloured or uncoloured, fluorescent or non-fluorescent chromophores such as those derived from optical brighteners, or chromophores derived from UVA and/or UVB screening agents, anti-ageing active agents or active agents intended for providing a benefit to the skin such as active agents having action on the barrier function, deodorant active agents other than mineral particles, antiperspirant active agents other than mineral particles, desquamating active agents, antioxidant active agents, moisturizing active agents, sebum-regulating active agents, active agents intended for limiting the sheen of the skin, active agents intended for combating the effects of pollution, antimicrobial or bactericidal active agents, antidandruff active agents, and fragrances.
the term “(hetero)aryl” means aryl or heteroaryl groups;
the term “(hetero)cycloalkyl” means cycloalkyl or heterocycloalkyl groups;
the “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆, and preferably C₁-C₄ alkyl radicals;
   an acylamino radical (—NR—COR′) in which the radical R is a hydrogen atom;
   a C₁-C₄ alkyl radical and the radical R′ is a C₁-C₄ alkyl radical; a carbamoyl radical ((R)₂N—CO—) in which the radicals R, which may be identical or different, represent a hydrogen atom or a C₁-C₄ alkyl radical;
   an alkylsulfonylamino radical (R'SO₂—NR—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxyl radical in the acid or salified form (preferably salified with an alkali metal or a substituted or unsubstituted ammonium);
   a cyano group (CN);
   a polyhalo(C₁-C₄)alkyl group, preferentially trifluoromethyl (CF₃);
the cyclic or heterocyclic part of a non-aromatic radical may be substituted with at least one substituent borne by a carbon atom, chosen from the groups:
   hydroxyl,
   C₁-C₄ alkoxy, C₂-C₄ (poly)hydroxyalkoxy,
   alkylcarbonylamino (RCO—NR′—), in which the radical R′ is a hydrogen atom or a C₁-C₄ alkyl radical and the radical R is a C₁-C₂ alkyl radical or an amino radical substituted with one or two identical or different C₁-C₄ alkyl groups;
   alkylcarbonyloxy (RCO—O—), in which the radical R is a C₁-C₄ alkyl radical or an amino radical substituted with one or two identical or different C₁-C₄ alkyl groups;
   alkoxycarbonyl ((RO—CO—) in which the radical R is a C₁-C₄ alkyl radical or an amino radical substituted with one or two identical or different C₁-C₄ alkyl groups;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
an “aryl” radical represents a monocyclic or fused or non-fused polycyclic hydrocarbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl, preferably phenyl;
a “heteroaryl” radical represents a monocyclic or fused or non-fused polycyclic, 5- to 22-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
a “cyclic” or “cycloalkyl” radical is a monocyclic or fused or non-fused polycyclic, non-aromatic cyclic hydrocarbon-based radical containing from 5 to 22 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl and morpholinyl;
an “alkyl” radical is a linear or branched, in particular C₁-C₆ and preferably C₁-C₄ saturated hydrocarbon-based radical;
an “alkenyl” radical is a linear or branched unsaturated hydrocarbon-based radical comprising one or more conjugated or non-conjugated double bonds;
an “alkynyl” radical is a linear or branched unsaturated hydrocarbon-based radical comprising one or more conjugated or non-conjugated triple bonds;
an “alkoxy radical” is an alkyl-oxy radical for which the alkyl radical is a linear or branched C₁-C₆ and preferentially C₁-C₄ hydrocarbon-based radical;
a “sugar” radical is a monosaccharide or polysaccharide radical, and the O-protected sugar derivatives thereof such as sugar esters of (C₁-C₆)alkylcarboxylic acids such as sugar esters of acetic acid, sugars containing amine group(s) and (C₁-C₄)alkyl derivatives, such as methyl derivatives, for instance methylglucose. Sugar radicals that may be mentioned include: sucrose, glucose, galactose, ribose, fucose, maltose, fructose, mannose, arabinose, xylose, lactose.
the term “monosaccharides” refers to a monosaccharide sugar comprising at least 5 carbon atoms of formula Cₓ(H₂O)ₓ with x an integer greater than or equal to 5, preferably x is greater than or equal to 6, in particular x is between 5 and 7 inclusive, preferably x=6, they may be of D or L configuration, and of alpha or beta anomer, and also the salts thereof and the solvates thereof such as hydrates;
the term “polysaccharides” refers to a polysaccharide sugar which is a polymer constituted of several saccharides bonded together via O-oside bonds, said polymers being constituted of monosaccharide units as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— where x is an integer greater than or equal to 5, preferably x is greater than or equal to 6, in particular x is between 5 and 7 inclusive and preferably x=6, and y is an integer which represents x−1, and w is an integer greater than or equal to 2, particularly of between 3 and 3000 inclusive, more particularly between 5 and 2500 and preferentially between 10 and 2300;
the term “sugar bearing amine group(s)” means that the sugar radical is substituted with one or more amino groups NR₁R₂ i.e. at least one of the hydroxyl groups of at least one saccharide unit of the sugar radical is replaced with a group NR₁R₂ with R₁ and R₂, which may be identical or different, representing i) a hydrogen atom, ii) a (C₁-C₆)alkyl group, iii) an aryl group such as phenyl, iv) an aryl(C₁-C₄)alkyl group such as benzyl, vii) —C(Y)—(Y′)_{f}R′₁ with Y and Y′, which may be identical or different, representing an oxygen atom, a sulfur atom or N(R′₂), preferably oxygen, f=0 or 1, preferably 0; and R′₁ and R′₂ representing i) to vi) of R₁ and R₂ defined previously, and in particular R′₁ denoting a (C₁-C₆)alkyl group such as methyl. Preferably, R₁ and R₂ represent a hydrogen atom or a (C₁-C₄)alkylcarbonyl group such as acetyl;
the term “organic or mineral acid salt” more particularly means organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{o}—C(O)—OH (I′), in which formula (I′) R^{o} represents a (hetero)aryl group such as phenyl, (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{o} preferably denoting a (C₁-C₆)alkyl group optionally substituted with 1, 2 or 3 hydroxyl or carboxyl groups; more preferentially, the monocarboxylic acids of formula (I′) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methanesulfonate or mesylate and ethanesulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate.
the “solvates” represent hydrates and also the combination with linear or branched C₁-C₄ alcohols such as ethanol, isopropanol or n-propanol.
the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “fluorescent chromophore” means a chromophore which is also capable of re-emitting in the visible range at an emission wavelength λₑₘ of between 400 and 800 nm, and higher than the absorption wavelength, preferably with a Stoke's shift, i.e. the difference between the maximum absorption wavelength and the emission wavelength is at least 10 nm. Preferably, fluorescent chromophores are derived from fluorescent dyes that are capable of absorbing in the visible range λ_{abs}, i.e. at a wavelength of between 400 and 800 nm, and of re-emitting in the visible range λₑₘ between 400 and 800 nm. More preferentially, fluorescent chromophores are capable of absorbing at a λ_{abs} of between 420 and 550 nm and of re-emitting in the visible range λₑₘ between 470 and 600 nm;
the term “optical brightening chromophore” means a chromophore derived from an optical brightening compound or “optical brighteners, optical brightening agents (OBAs)” or “fluorescent brightening agents (FBAs)” or “fluorescent whitening agents (FWAs)”, i.e. agents which absorb UV radiation, i.e. at a wavelength λ_{abs} of between 250 and 350 nm, and of subsequently re-emitting this energy by fluorescence in the visible range at an emission wavelength λₑₘ of between 400 and 600 nm, i.e. wavelengths between blue-violet and blue-green with a maximum in the blue range. Optical brightening chromophores are thus colourless to the eye;
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm);
the term “UV-8 screening agent” means a chromophore derived from a compound which screens out (or absorbs) UV-B ultraviolet rays at a wavelength of between 280 and 320 nm.
   Furthermore, unless otherwise indicated, the limits delimiting the extent of a range of values are included in that range of values.

### a) The PHA Copolymer(s)

The composition of the invention comprises as first ingredient a) one or more PHA copolymers which contain, or preferably consist of, at least two different repeating polymer units chosen from the units (A) and (B) as defined previously.

The term “copolymer” means that said polymer is derived from the polycondensation of repeating polymer units that are different from each other, i.e. said polymer is derived from the polycondensation of repeating polymer units (A) with (B), it being understood that the polymer units (A) are different from the polymer units (B).

According to a particular embodiment of the invention, the PHA copolymer(s) consist of two different repeating polymer units chosen from the units (A) and (B) as defined previously.

More particularly, the PHA copolymer(s) according to the invention comprise the repeating unit of formula (I), and also the optical or geometrical isomers thereof, the organic or mineral acid or base salts thereof, and the solvates thereof such as hydrates:

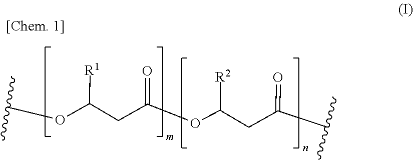

in which formula (I):
R¹ and R² are as defined previously,
m and n are integers greater than or equal to 1, preferably the sum n+m is inclusively between 450 and 1400,
preferably m<n.

According to a particular embodiment, the PHA copolymer(s) of composition a) contain three different repeating polymer units (A), (B) and (C), and preferably consist of three different polymer units (A), (B) and (C) below, and also the optical or geometrical isomers thereof and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

in which polymer units (A), (B) and (C):
R¹ and R² are as defined previously;
R³ represents a cyclic or non-cyclic, linear or branched, saturated or unsaturated hydrocarbon-based group comprising from 1 to 30 carbon atoms;
optionally substituted with one or more atoms or groups a) to l) and/or optionally interrupted with one or more heteroatoms or groups a′) to c′) as defined for R¹; in particular represents a hydrocarbon-based group chosen from substituted and/or interrupted, linear or branched (C₁-C₂₈)alkyl and linear or branched (C₂-C₂₈)alkenyl, in particular a linear hydrocarbon-based group, more particularly (C₄-C₂₀)alkenyl; preferably, the hydrocarbon-based group has a carbon number corresponding to the number of carbon atoms of the radical R¹, or else corresponding to the number of carbon atoms of the radical R¹ from which at least three carbon atoms are subtracted, preferably corresponding to the number of carbon atoms of the radical R¹ from which four carbon atoms are subtracted; and
   it being understood that:
(A) is different from (B) and (C), (B) is different from (A) and (C), and (C) is different from (A) and (B); and
preferably, the molar percentage of units (A) is less than the molar percentage of units (B) and less than the molar percentage of units (C) notably if R² represents an alkyl group and/or R³ represents an alkyl group; preferably, R³ represents an alkyl group with a carbon number corresponding to the carbon number of R² from which two carbon atoms are subtracted.

According to a particular embodiment of the invention, the PHA copolymer(s) comprise the repeating unit of formula (II), and also the optical or geometrical isomers thereof, the organic or mineral acid or base salts thereof, and the solvates thereof such as hydrates:

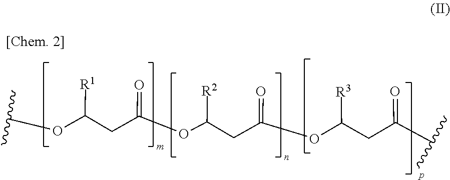

in which formula (II):
R¹, R² and R³ are as defined previously;
m, n and p are integers greater than or equal to 1; preferably, the sum n+m+p is inclusively between 450 and 1400; and
preferably, m<n+p, preferably R³ represents an alkyl group with a carbon number corresponding to the carbon number of R² from which two carbon atoms are subtracted.

According to a particular embodiment, the PHA copolymer(s) of composition a) contain four different repeating polymer units (A), (B), (C) and (D), and preferably consist of four different polymer units (A), (B), (C) and (D), below, and also the optical or geometrical isomers thereof, the organic or mineral acid or base salts thereof, and the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)

in which polymer units (A), (B), (C) and (D):
R¹, R² and R³ are as defined previously;
R⁴ represents a cyclic or non-cyclic, linear or branched, saturated hydrocarbon-based group comprising from 3 to 30 carbon atoms optionally substituted with one or more atoms or groups a) to l) and/or optionally interrupted with one or more heteroatoms or groups a′) to c′) as defined for R¹; it in particular represents a hydrocarbon-based group chosen from linear or branched (C₄-C₂₈)alkyl optionally substituted with one or more atoms or groups a) to l) and/or interrupted with one or more heteroatoms or groups a′) to c′) as defined for R¹; and
   it being understood that:
(A) is different from (B), (C) and (D), (B) is different from (A), (C) and (D), (C) is different from (A), (B) and (D), and (D) is different from (A), (B) and (C); and
preferably the molar percentage of units (A) is less than the molar percentage of units (B) and less than the molar percentage of units (C), notably if R² represents an alkyl group and/or R³ represents an alkyl group; and R⁴ represents a substituted and/or interrupted alkyl, optionally substituted and/or optionally interrupted alkenyl or optionally substituted and/or optionally interrupted alkynyl group; preferably R³ represents an alkyl group with a carbon number corresponding to the carbon number of R² from which two carbon atoms are subtracted and R⁴ represents an optionally substituted and/or interrupted alkyl, optionally substituted and/or interrupted alkenyl or optionally substituted and/or interrupted alkynyl group with a carbon number corresponding to the carbon number of R¹ from which two carbon atoms are subtracted.

According to a particular embodiment of the invention, the PHA copolymer(s) comprise the repeating unit of formula (III), and also the optical or geometrical isomers thereof, the organic or mineral acid or base salts thereof, and the solvates thereof such as hydrates:

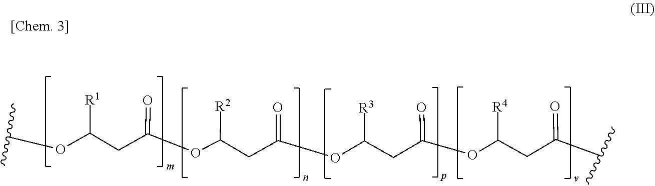

in which formula (III):
R¹, R², R³ and R⁴ are as defined previously;
m, n, p and v are integers greater than or equal to 1;
preferably, the sum n+m+p+v is inclusively between 450 and 1400; and
preferably, n>m+v; more preferentially n+p>m+v—preferably R³ represents an alkyl group with a carbon number corresponding to the carbon number of R² from which two carbon atoms are subtracted and R⁴ represents an optionally substituted and/or optionally interrupted alkenyl or substituted and/or optionally interrupted alkynyl group with a carbon number corresponding to the carbon number of R¹ from which two carbon atoms are subtracted.

According to a more particular embodiment, the PHA copolymer(s) of composition a) contain five different repeating polymer units (A), (B), (C), (D) and (E), and preferably consist of five different polymer units (A), (B), (C), (D) and (E), below, and also the optical or geometrical isomers thereof, the organic or mineral acid or base salts thereof, and also the solvates thereof such as hydrates:

—[—O—CH(R¹)—CH₂—C(O)—]— unit (A)

—[—O—CH(R²)—CH₂—C(O)—]— unit (B)

—[—O—CH(R³)—CH₂—C(O)—]— unit (C)

—[—O—CH(R⁴)—CH₂—C(O)—]— unit (D)

—[—O—CH(R⁵)—CH₂—C(O)—]— unit (E)

in which polymer units (A), (B), (C), (D) and (E):
R¹, R², R³ and R⁴ are as defined previously;
and
R⁵ represents a cyclic or non-cyclic, linear or branched, saturated hydrocarbon-based group comprising from 3 to 30 carbon atoms optionally substituted with one or more atoms or groups a) to l) and/or optionally interrupted with one or more heteroatoms or groups a′) to c′) as defined for R¹; it in particular represents a hydrocarbon-based group chosen from linear or branched (C₄-C₂₈)alkyl, optionally substituted with one or more atoms or groups a) to l) and/or interrupted with one or more heteroatoms or groups a′) to c′) as defined for R¹; preferably, the hydrocarbon-based group has a carbon number corresponding to the number of carbon atoms of the radical R⁴ from which at least one carbon atom is subtracted, preferably corresponding to the number of carbon atoms of the radical R⁴ from which at least two carbon atoms are subtracted, preferably from which two carbon atoms are subtracted;
   it being understood that:
(A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D); and
preferably, the molar percentage of units (A) is less than the molar percentage of units (B) and less than the molar percentage of units (C), notably if R² represents an alkyl group and/or R³ represents an alkyl group, and R⁴ and R⁵ represent an optionally substituted and/or interrupted alkyl, optionally substituted and/or optionally interrupted alkenyl or optionally substituted and/or optionally interrupted alkynyl group; preferably R³ represents an alkyl group with a carbon number corresponding to the carbon number of R² from which two carbon atoms are subtracted, and R⁴ represents an optionally substituted and/or optionally interrupted alkenyl or optionally substituted and/or optionally interrupted alkynyl group with a carbon number corresponding to the carbon number of R¹ from which two carbon atoms are subtracted, and R⁵ represents an optionally substituted and/or optionally interrupted alkenyl or optionally substituted and/or optionally interrupted alkynyl group with a carbon number corresponding to the carbon number of R¹ from which four carbon atoms are subtracted.

According to a particular embodiment of the invention, the PHA copolymer(s) comprise the repeating unit of formula (IV), and also the optical or geometrical isomers thereof, the organic or mineral acid or base salts thereof, and the solvates thereof such as hydrates:

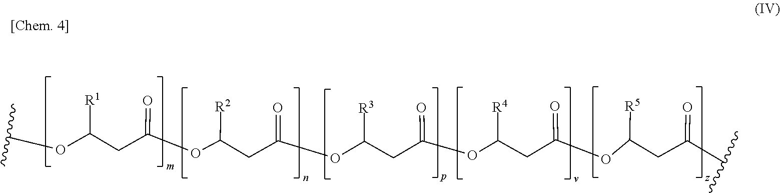

in which formula (IV):
R¹, R², R³, R⁴ and R⁵ are as defined previously;
m, n, p, v and z are integers greater than or equal to 1; preferably, the sum n+m+p+v+z is inclusively between 450 and 1400; and
preferably, when R¹ represents a substituted and/or interrupted alkyl, optionally substituted and/or optionally interrupted alkenyl or optionally substituted and/or optionally interrupted alkynyl group, R² and R³ represent an alkyl group, and the groups R⁴ and R⁵ represent an optionally substituted and/or optionally interrupted alkyl, optionally substituted and/or optionally interrupted alkenyl or optionally substituted and/or optionally interrupted alkynyl group, then n>m+v+z; more preferentially n+p>m+v+z; preferably R³ represents an alkyl group with a carbon number corresponding to the carbon number of R² from which two carbon atoms are subtracted, and R⁴ represents an optionally substituted and/or optionally interrupted alkenyl or optionally substituted and/or optionally interrupted alkynyl group with a carbon number corresponding to the carbon number of R¹ from which two carbon atoms are subtracted, and R⁵ represents an optionally substituted and/or optionally interrupted alkenyl or optionally substituted and/or optionally interrupted alkynyl group with a carbon number corresponding to the carbon number of R¹ from which four carbon atoms are subtracted.

Preferably, R¹ represents a linear or branched, preferably linear, (C₅-C₂₈)alkyl hydrocarbon-based chain. According to one embodiment of the composition according to the invention, the PHA copolymer(s) are such that the radical R¹ is a substituted alkyl group comprising 5 to 14 and preferably between 6 and 12 carbon atoms, more preferentially between 7 and 10 carbon atoms such as n-pentyl, n-hexyl, n-octyl or n-nonyl.

According to another embodiment, the hydrocarbon-based chain of the radical R¹ of the invention is 1) either substituted, 2) or interrupted, 3) or substituted and interrupted.

According to a particular embodiment of the invention, the PHA copolymer(s) are such that R¹ represents a hydrocarbon-based chain, notably an alkyl group as defined previously, which is interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably, R¹ represents an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆. Preferably, said interrupted hydrocarbon-based chain, notably alkyl, is linear.

According to another embodiment of the invention, the PHA copolymer(s) are such that R¹ represents a hydrocarbon-based chain, notably an alkyl group as defined previously, substituted with one or more (preferably one) atoms or groups chosen from: a) to k) as defined previously. Preferably, said hydrocarbon-based chain is substituted with only one atom or group chosen from: a) to k) as defined previously.

According to a particular embodiment of the invention, the PHA copolymer(s) are such that R¹ represents a hydrocarbon-based chain, notably an alkyl group as defined previously, which is substituted with one or more (preferably one) groups chosen from b) hydroxyl, c) thiol, d) (di)(C₁-C₄)(alkyl)amino and preferably amino, e) carboxyl, i) (hetero)cycloalkyl such as anhydride, or epoxide, j) a cosmetic active agent such as coloured or uncoloured, fluorescent or non-fluorescent chromophores such as optical brighteners, UV-screening agents, anti-ageing active agents, h) (hetero)aryl such as phenyl or furyl, k) R—X with R representing a group chosen from α) cycloalkyl such as cyclohexyl, β) heterocycloalkyl such as a sugar radical, preferably a monosaccharide such as glucose, γ) (hetero)aryl such as phenyl, δ) an organic active agent as defined previously and X representing a′) O, S, N(Rₐ), b′) carbonyl, c′) or combinations thereof of a′) with b′) such as ester, amide or urea; Rₐ represents a hydrogen atom or a (C₁-C₄)alkyl or aryl(C₁-C₄)alkyl group such as benzyl, preferably Rₐ represents a hydrogen atom.

Even more preferentially, the PHA copolymer(s) are such that R¹ represents a hydrocarbon-based chain, notably an alkyl group as defined previously, which is substituted with one or more (preferably one) groups chosen from b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom.

Preferably, said substituted hydrocarbon-based chain, notably alkyl, is linear.

According to another particular embodiment of the invention, the hydrocarbon-based chain of the radical R1 of the invention is substituted and interrupted.

According to a particular embodiment of the invention, the hydrocarbon-based chain (notably an alkyl group as defined previously) of the radical R¹ of the invention is:
substituted with one or more (preferably one) groups chosen from b) hydroxyl, c) thiol, d) (di)(C₁-C₄)(alkyl)amino and preferably amino, e) carboxyl, i) (hetero)cycloalkyl such as anhydride, or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, δ) a cosmetic active agent as defined previously and X representing a′) O, S, N(Rₐ), b′) carbonyl, c′) or combinations thereof of a′) with b′) such as ester, amide or urea; Rₐ representing a hydrogen atom or a (C₁-C₄)alkyl or aryl(C₁-C₄)alkyl group such as benzyl, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆.

According to a preferred embodiment of the invention, the hydrocarbon-based chain (notably an alkyl group as defined previously) of the radical R¹ of the invention is:
substituted with one or more (preferably one) groups chosen from b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆.

Preferably, said substituted and interrupted hydrocarbon-based chain, notably alkyl, is linear.

More preferentially, when said hydrocarbon-based chain R¹ is substituted, it is substituted at the end of the chain on the opposite side from the carbon atom which bears said radical R¹.

According to one embodiment of the invention, said hydrocarbon-based chain R¹ has the following formula —(CH₂)ᵣ—X-(ALK)ᵤ-G with X being as defined previously, in particular representing O, S or N(Rₐ), preferably S, ALK represents a linear or branched, preferably linear, (C₁-C₁₀)alkylene and more particularly (C₁-C₈)alkylene chain, r represents an integer inclusively between 6 and 11, preferably between 7 and 10 such as 8; u is equal to 0 or 1; and G represents a hydrogen atom or a group chosen from hydroxyl, carboxyl, (di)(C₁-C₄)(alkyl)amino, (hetero)aryl in particular aryl such as phenyl, cycloalkyl such as cyclohexyl, or a sugar, in particular a monosaccharide optionally protected with one or more groups such as acyl, preferably Sug represents:

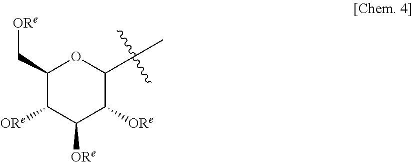

with R^{e} representing a group R^{f}-c(O)—, with R^{f} representing a (C₁-C₄) alkyl group such as methyl. Preferably, when u is equal to 0, G represents a cycloalkyl such as cyclohexyl or a sugar as defined previously. According to another advantageous variant, when u is equal to 1, G represent a hydrogen atom or a group chosen from hydroxyl, carboxyl, (di)(C₁-C₄)(alkyl)amino or (hetero)aryl, in particular aryl such as phenyl.

According to another particular embodiment of the invention, the PHA copolymer(s) are such that R¹ represents (C₃-C₃₀)alkyl substituted with one or more halogen atoms such as fluorine, chlorine or bromine, more particularly linear (C₄-C₂₀)alkyl, even more particularly (C₅-C₁₃)alkyl, substituted with a halogen atom such as bromine. Preferably, the halogen atom is substituted at the end of said alkyl group. More preferentially, R¹ represents 1-halo-5-yl such as 1-bromo-5-yl.

According to another particular embodiment of the invention, the PHA copolymer(s) are such that R¹ represents a (C₃-C₃₀)alkyl group substituted with one or more groups chosen from a) cyano, and more particularly represents a (C₃-C₁₃)alkyl group, which is preferably linear, substituted with a cyano group, such as 1-cyano-3-propyl.

According to another particular embodiment of the invention, the PHA copolymer(s) are such that R¹ represents vii) a (hetero)aryl(C₁-C₂)alkyl and more particularly aryl(C₁-C₂)alkyl group, preferably phenylethyl.

According to another particular embodiment of the invention, the PHA copolymer(s) are such that R¹ represents a (C₃-C₃₀)alkyl group substituted with one or more groups chosen from c) (hetero)cycloalkyl. More particularly, R¹ represents a (C₅-C₁₃)alkyl group, which is preferably linear, substituted with a heterocycloalkyl group such as epoxide.

In particular, the PHA copolymer(s) are such that R² is chosen from linear or branched (C₁-C₂₈)alkyl, and linear or branched (C₂-C₂₈)alkenyl, in particular a linear hydrocarbon-based group, particularly (C₃-C₂₀)alkyl or (C₃-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted.

According to one embodiment of the invention, the PHA copolymer(s) are such that the radical R² is a linear or branched, preferably linear, (C₁-C₈)alkyl, in particular (C₂-C₆)alkyl, preferably (C₄-C₆)alkyl group such as n-pentyl or n-hexyl.

According to another embodiment of the composition according to the invention, the PHA copolymer(s) comprise a branched (C₃-C₈)alkyl, particularly (C₄-C₆)alkyl radical R², preferably a branched (C₄-C₅)alkyl radical such as isobutyl.

According to another embodiment of the composition according to the invention, the PHA copolymer(s) of the invention comprise the units (A) bearing an alkyl radical R¹ as defined previously, the units (B) as defined previously and the units (C) bearing a linear or branched (C₆-C₂₀)alkenyl, particularly (C₇-C₁₄)alkenyl and more particularly (C₈-C₁₀)alkenyl radical, which is preferably linear and comprising only one unsaturation at the chain end, in particular, —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5.

According to one embodiment of the composition according to the invention, the PHA copolymer(s) comprise units (A) bearing an alkyl radical R¹ comprising between 8 and 16 carbon atoms substituted with one or more (preferably one) groups chosen from hydroxyl, (di)(C₁-C₄)(alkyl)amino, carboxyl, and R—X— as defined previously, preferably R—S— with R representing a cycloalkyl group such as cyclohexyl, heterocycloalkyl such as a sugar, more preferentially a monosaccharide such as glucose, optionally substituted aryl(C₁-C₄)alkyl such as (C₁-C₄)(alkyl)benzyl or phenylethyl, or heteroaryl(C₁-C₄)alkyl such as furylmethyl.

According to one embodiment of the composition according to the invention, the copolymer(s) comprise units B bearing a linear or branched, preferably linear, (C₁-C₈)alkyl, particularly (C₂-C₆)alkyl, preferably (C₄-C₅)alkyl radical R² such as pentyl.

According to another embodiment of the composition according to the invention, the PHA copolymer(s) comprise units (A) containing an alkyl radical R¹ as defined previously, units (B) as defined previously and units (C) containing a linear or branched (C₆-C₂₀)alkenyl, particularly (C₇-C₁₄)alkenyl radical and more particularly (C₈-C₁₀)alkenyl radical, which is preferably linear, and comprising only one unsaturation at the chain end such as —[CH₂]_{q}—CH═CH₂ and p represents an integer inclusively between 3 and 8, preferably between 4 and 6, such as 5.

According to a particular embodiment of the invention, in the PHA copolymer(s), the unit (A) comprises a hydrocarbon-based chain R¹ which is a substituted and/or interrupted alkyl, substituted and/or interrupted alkenyl, or substituted and/or interrupted alkynyl group, as defined previously, said unit (A) is present in a molar percentage ranging from 0.1% to 50%, more preferentially a molar percentage ranging from 0.5% to 40%, even more preferentially a molar percentage ranging from 1% to 40%, better still a molar percentage ranging from 2% to 30%, or a molar percentage ranging from 5% to 20%.

Preferably, when R¹ of the unit (A) is a substituted and/or interrupted alkyl, substituted and/or interrupted alkenyl or substituted and/or interrupted alkynyl hydrocarbon-based chain, said unit (A) is present in a molar percentage of less than or equal to 30%, more particularly less than 20%, preferably between 8% and 13%.

According to a more particular embodiment of the invention in the PHA copolymer(s), the unit (A) is present in a molar percentage ranging from 0.1 mol % to 99 mol %, preferentially a molar percentage ranging from 0.5 mol % to 50 mol %, more preferentially a molar percentage ranging from 1 mol % to 40 mol %, even more preferentially a molar percentage ranging from 2 mol % to 30 mol %, better a molar percentage ranging from 5 mol % to 20 mol %; better still a molar percentage ranging from 10 mol % to 30 mol % of units (A); the unit (B) is present in a molar percentage ranging from 1 mol % to 40 mol %; preferentially a molar percentage from 2 mol % to 10 mol %, more preferentially a molar percentage ranging from 5 mol % to 35 mol % of units (B); and/or the unit (C) is present in a molar percentage ranging from 0.5 mol % to 20 mol %, preferentially a molar percentage ranging from 1 mol % to 7 mol %, more preferentially from 0.5 mol % to 7 mol % of units (C).

According to a more particular embodiment of the invention in the PHA copolymer(s), the unit (A) is present in a molar percentage ranging from 0.1% to 50%, more preferentially a molar percentage ranging from 0.5% to 40%, even more preferentially a molar percentage ranging from 1% to 40%, better still a molar percentage ranging from 5% to 30%, a molar percentage ranging from 8% to 20%; the unit (B) is present in a molar percentage ranging from 70% to 99.5%, preferably between 60% and 95%; and the unit (C) is present in a molar percentage ranging from 0% to 30%, preferably between 1% and 25%, more preferentially between 5% and 24%, relative to the sum total, the unit (D) is present in a molar percentage ranging from 0% to 10%, preferably between 0.1% and 5%, more preferentially between 0.5% and 2% relative to the sum total and the unit (E) 0% to 10%, preferably between 0.1% and 5%, more preferentially between 0.5% and 2% relative to the sum total and the unit. Advantageously, the PHA copolymer(s) of the invention comprise from 70 mol % to 90 mol % of units (B); and from 6 mol % to 24 mol % of units (C).

The values of the molar percentages of the units (A), (B) and (C) of the PHA copolymer(s) are calculated relative to the total number of moles of (A)+(B) if the copolymer(s) do not comprise any additional units (C); otherwise, if the copolymer(s) of the invention contain three different units (A), (B) and (C), then the molar percentage is calculated relative to the total number of moles (A)+(B)+(C); otherwise, if the copolymer(s) of the invention contain four different units (A), (B), (C) and (D), then the molar percentage is calculated relative to the total number of moles (A)+(B)+(C)+(D); otherwise, if the copolymer(s) of the invention contain four different units (A), (B), (C), (D) and (E), then the molar percentage is calculated relative to the total number of moles (A)+(B)+(C)+(D)+(E).

Preferentially, the PHA copolymer(s) of the invention comprise the following repeating units, and also the optical or geometrical isomers thereof, the organic or mineral acid or base salts thereof, and the solvates thereof such as hydrates:

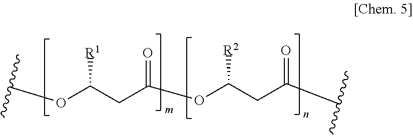

| Com- | | |
|---|---|---|
| pounds | R¹ | R² |
| (1) | —(CH₂)₈—S—CH(CH₃)—C(O)—OH | —(CH₂)₄—CH₃ |
| (2) | —(CH₂)₅—S—(CH₂) [symbol] —CH₃ | —(CH₂)₄—CH₃ |
| (3) | —(CH₂)₈—S—(CH₂)₈—OH | —(CH₂)₄—CH₃ |
| (4) | —(CH₂)₈—S—(CH₂)₂—NH₂ | —(CH₂)₄—CH₃ |
| (5) | —(CH₂) [symbol] —S-Cycl | —(CH₂)₄—CH₃ |
| (6) | —(CH₂)₅—S—CH₂-Fur | —(CH₂)₄—CH₃ |
| (7) | —(CH₂) [symbol] —S-Sug | —(CH₂)₄—CH₃ |
| (8) | —(CH₂)₈—S—(CH₂)₂-Ar | —(CH₂)₄—CH₃ |
| (9) | —(CH₂)₈—S—CH₂-Ar′ | —(CH₂)₄—CH₃ |
| (10) | —(CH₂)₈—S—CH(CH₃)—C(O)—OH | —(CH₂)₅—CH₃ |
| (11) | —(CH₂)₅-Hal | —(CH₂)₅—CH₃ |
| (16) | —(CH₂)₅—CH₃ | —(CH₂)₃—CH₃ |
| (17) | —(CH₂) [symbol] —CH₃ | —(CH₂)₄—CH₃ |
| (18) | —(CH₂) [symbol] —CH₃ | —(CH₂)₆—CH₃ |
| (19) | —(CH₂)₃—CH(CH₃)CH₃ | —CH₂—CH(CH₃)CH₃ |
| (20) | —(CH₂) [symbol] —CH═CH₂ | —(CH₂)₅—CH₃ |
| (21) | —(CH₂)₂—CH═C(CH₃)CH₃ | —CH₂—CH(CH₃)CH₃ |

[symbol]

indicates data missing or illegible when filed

m and n are as defined previously, Hal represents a halogen atom such as bromine and t represents an integer between 1 and 10, preferably between 3 and 8 such as 6.
Ar: represents a (hetero)aryl group such as phenyl;
Ar′: represents a (C₁-C₄)alkyl(hetero)aryl group such as t-butylphenyl, preferably 4-t-butylphenyl;
Cycl: represents a cyclohexyl group;
Fur: represents a furyl group, preferably 2-furyl;
Sug: represents a sugar group, in particular a monosaccharide optionally protected with one or more groups such as acyl; preferably, Sug represents:

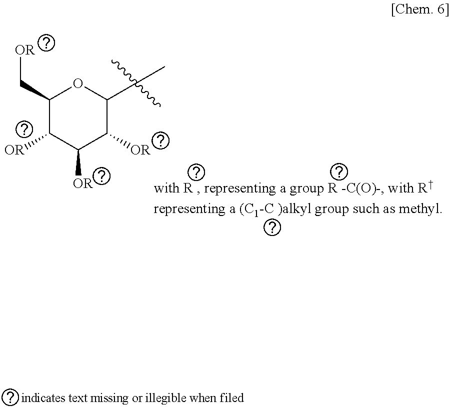

In particular, the stereochemistry of the carbon atoms bearing the radicals R¹ and R² is of the same (R) or (S) configuration, preferably of (R) configuration.
More particularly, the stereochemistry of the carbon atoms bearing the radicals R¹, R² and R³ is of the same (R) or (S) configuration, preferably of (R) configuration.
More particularly, the stereochemistry of the carbon atoms bearing the radicals R¹, R², R³ and R⁴ is of the same (R) or (S) configuration, preferably of (R) configuration.
More particularly, the stereochemistry of the carbon atoms bearing the radicals R¹, R², R³, R⁴ and R⁵ is of the same (R) or (S) configuration, preferably of configuration.

More preferentially, the PHA copolymer(s) have the following formula, and also the optical isomers thereof, the organic or mineral acid or base salts thereof, and the solvates thereof such as hydrates:

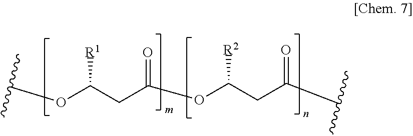

| Compounds | R¹ | R² |
|---|---|---|
| (1′) | -(CH₂)₈-S-CH(CH₃)-C(O)-OH | -(CH₂)₄-CH₃ |
| (2′) | -(CH₂)₈-S-(CH₂)₇-CH2 | -(CH₂)₄-CH₃ |
| (3′) | -(CH₂)₈-S-(CH₂)₈-OH | -(CH₂)₄-CH₃ |
| (4′) | -(CH₂)₈-S-(CH2)₂-NH₂ | -(CH₂)₄-CH₃ |
| (5′) | -(CH₂)₈-S-Cycl | -(CH₂)₄-CH₃ |
| (6′) | -(CH₂)₈-S-CH₂-Fur | -(CH₂)₄-CH₃ |
| (7′) | -(CH₂)₈-S-Sug | -(CH₂)₄-CH₃ |
| (8′) | -(CH₂)₈-S-(CH₂)₂-Ar | -(CH₂)₄-CH₃ |
| (9′) | -(CH₂)₈-S-CH₂-Ar | -(CH₂)₄-CH₃ |
| (10′) | -(CH₂)₈-S-CH(CH₃)-C(O)-OH | -(CH₂)₈-CH₃ |
| (11′) | -(CH₂)₈-Hal | -(CH₂)₈-CH₃ |
| (12′) | -(CH₂)₃-CN | -(CH₂)₈-CH₃ |
| (13′) | 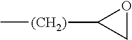 | -(CH₂)₈-CH₃ |
| (14′) | -(CH₂)₂-Ar | -(CH₂)₈-CH₃ |

M, n, Hal, t, Ar, Ar′, Cycl, Fur and Sug are as defined previously for compounds (1) to (14).

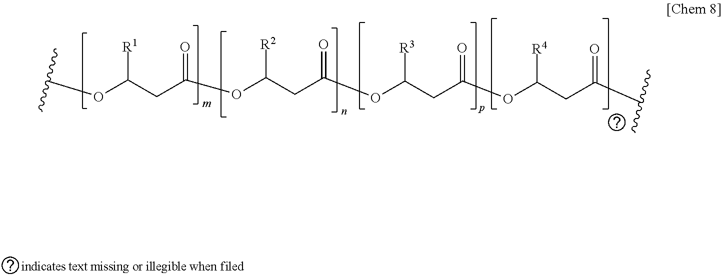

| Com- | | | | |
|---|---|---|---|---|
| pounds | R¹ | R² | R³ | R⁴ |
| (15) | —(CH₂)₃—CN | —(CH₂)₅—CH₃ | —(CH₂)₃—CH₃ | —CN |
| (16) | —(CH₂)₂-Ar | —(CH₂)₅—CH₃ | —(CH₂)₃—CH₃ | -Ar |

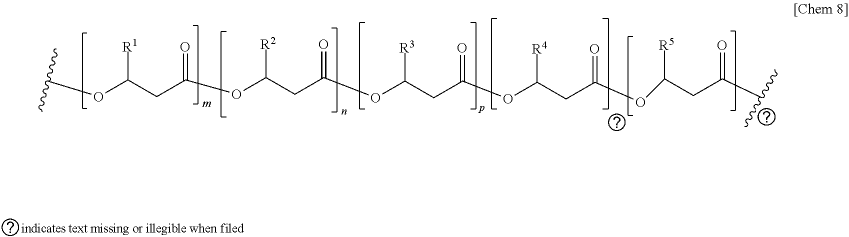

| Compounds | R¹ | R² | R³ | R⁴ | R⁵ |
|---|---|---|---|---|---|
| 17 | -(CH₂)₈-S-CH(CH₃)- | -(CH₂)₄- | -(CH₂)₂- | -(CH₂)₈-S-CH(CH₃)- | -(CH₂)₈-S-CH(CH₃)- |
| | C(O)-OH | CH₃ | CH₃ | C(O)-OH | C(O)-OH |
| 18 | -(CH₂)₈-S-(CH₃)₇- | -(CH₂)₄- | -(CH₂)₂- | -(CH₂)₈-S-(CH₃)₇- | -(CH₂)₈-S-(CH₃)₇- |
| | CH₃ | CH₃ | CH₃ | CH₃ | CH₃ |
| 19 | -(CH₂)₈-S-(CH₂)₈-OH | -(CH₂)₄- | -(CH₂)₂- | -(CH₂)₈-S-(CH₂)₈-OH | -(CH₂)₈-S-(CH₂)₈-OH |
| | | CH₃ | CH₃ | | |
| 20 | -(CH₂)₈-S-(CH₂)₂- | -(CH₂)₄- | -(CH₂)₂- | -(CH₂)₈-S-(CH₂)₂- | -(CH₂)₈-S-(CH₂)₂- |
| | NH₂ | CH₃ | CH₃ | NH₂ | NH₂ |
| 21 | -(CH₂)₈-S-Cycl | -(CH₂)₄- | -(CH₂)₂- | -(CH₂)₈-S-Cycl | -(CH₂)₈-S-Cycl |
| | | CH₃ | CH₃ | | |
| 22 | -(CH₂)₈-S-CH₂-Fur | -(CH₂)₄- | -(CH₂)₂- | -(CH₂)₈-S-CH₂-Fur | -(CH₂)₈-S-CH₂-Fur |
| | | CH₃ | CH₃ | | |
| 23 | -(CH₂)₈-S-Sug | -(CH₂)₄- | -(CH₂)₂- | -(CH₂)₈-S-Sug | -(CH₂)₈-S-Sug |
| | | CH₃ | CH₃ | | |
| 24 | -(CH₂)₈-S-(CH₂)₂-Ar | -(CH₂)₄- | -(CH₂)₂- | -(CH₂)₈-S-(CH₂)₂-Ar | -(CH₂)₈-S-(CH₂)₂-Ar |
| | | CH₃ | CH₃ | | |
| 25 | -(CH₂)₈-S-CH₂-Ar′ | -(CH₂)₄- | -(CH₂)₂- | -(CH₂)₈-S-CH₂-Ar′ | -(CH₂)₈-S-CH₂-Ar′ |
| | | CH₃ | CH₃ | | |
| 26 | -(CH₂)₈-S-CH(CH₃)- | -(CH₂)₄- | -(CH₂)₂- | -(CH₂)₈-S-CH(CH₃)- | -(CH₂)₈-S-CH(CH₃)- |
| | C(O)-OH | CH₃ | CH₃ | C(O)-OH | C(O)-OH |
| 27 | -(CH₂)₈-Hal | -(CH₂)₄- | -(CH₂)₂- | -(CH₂)₈-Hal | -(CH₂)₈-Hal |
| | | CH₃ | CH₃ | | |
| 28 | 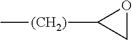 | -(CH₂)₄- | -(CH₂)₂- | 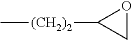 | 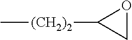 |
| | | CH₃ | CH₃ | | |

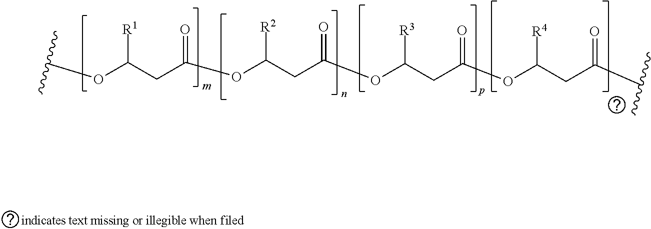

| Com- | | | | |
|---|---|---|---|---|
| pounds | R¹ | R² | R³ | R⁴ |
| (25) | —(CH₂)₆—CH₃ | —(CH₂)₅—CH₃ | —(CH₂)₄—CH₃ | —(CH₂)—CH₃ |
| (26) | —(CH₂)₆—CH₃ | —(CH₂)₄—CH₃ | —(CH₂)₂—CH₃ | —CH₃ |
| (27) | —(CH₂)₂—CN | —(CH₂)₅—CH₃ | —(CH₂)₃—CH₃ | —CN |
| (28) | —(CH₂)₂-Ar | —(CH₂)₅—CH₃ | —(CH₂)₅—CH₃ | -Ar |

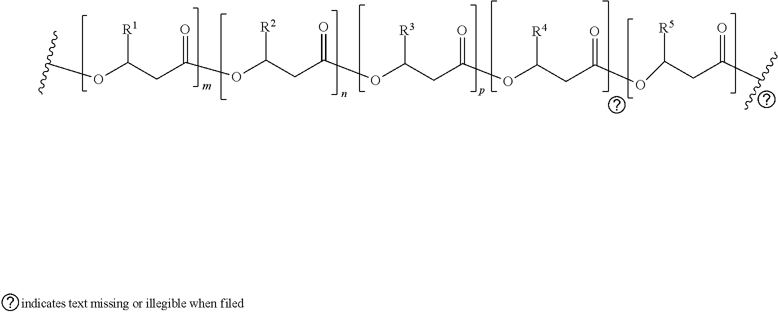

| Compounds | R¹ | R² | R³ | R⁴ | R⁵ |
|---|---|---|---|---|---|
| (29) | -(CH₂)₈-CH=CH₂ | -(CH₂)₄- | -(CH₂)₂- | -(CH₂)₈-CH=CH₂ | -(CH₂)₈-CH=CH₂ |
| | | CH₃ | CH₃ | | |
| (30) | -(CH₂)₈-CH=CH₂ | -(CH₂)₄- | -(CH₂)₂- | -(CH₂)₈-CH=CH₂ | -(CH₂)₈-CH=CH₂ |
| | | CH₃ | CH₃ | | |
| (31) | -(CH₂)₈-CH₃ | -(CH₂)₄- | -(CH₂)₂- | -(CH₂)₈-CH₃ | -CH₃ |
| | | CH₃ | CH₃ | | |
| (32) | -(CH₂)₁₀-CH₃ | -(CH₂)₄- | -(CH₂)₂- | -(CH₂)₁₀-CH₃ | -(CH₂)₁₀-CH₃ |
| | | CH₃ | CH₃ | | |
| (33) | -CH₂)₈-S-CH(CH₃)- | -(CH₂)₄- | -(CH₂)₂- | -CH₂)₈-S-CH(CH₃)- | -CH₂)₈-S-CH(CH₃)- |
| | C(O)-OH | CH₃ | CH₃ | C(O)-OH | C(O)-OH |
| (34) | -(CH₂)₈-S-(CH₃)₇- | -(CH₂)₄- | -(CH₂)₂- | -(CH₂)₈-S-(CH₃)₇- | -(CH₂)₈-S-(CH₃)₇- |
| | CH₃ | CH₃ | CH₃ | CH₃ | CH₃ |
| (35) | -(CH₂)₈-S-(CH₂)₈-OH | -(CH₂)₄- | -(CH₂)₂- | -(CH₂)₈-S-(CH₂)₈-OH | -(CH₂)₈-S-(CH₂)₈-OH |
| | | CH₃ | CH₃ | | |
| (36) | -(CH₂)₈-S-(CH₂)₂- | -(CH₂)₄- | -(CH₂)₂- | -(CH₂)₈-S-(CH₂)₂- | -(CH₂)₈-S-(CH₂)₂- |
| | NH₂ | CH₃ | CH₃ | NH₂ | NH₂ |
| (37) | -(CH₂)₈-S-Cycl | -(CH₂)₄- | -(CH₂)₂- | -(CH₂)₈-S-Cycl | -(CH₂)₈-S-Cycl |
| | | CH₃ | CH₃ | | |
| (38) | -(CH₂)₈-S-CH₂-Fur | -(CH₂)₄- | -(CH₂)₂- | -(CH₂)₈-S-CH₂-Fur | -(CH₂)₈-S-CH₂-Fur |
| | | CH₃ | CH₃ | | |
| (39) | -(CH₂)₈-S-Sug | -(CH₂)₄- | -(CH₂)₂- | -(CH₂)₈-S-Sug | -(CH₂)₈-S-Suo |
| | | CH₃ | CH₃ | | |
| (40) | -(CH₂)₈-S-(CH₂)₂-Ar | -(CH₂)₄- | -(CH₂)₂- | -(CH₂)₈-S-(CH₂)₂-Ar | -(CH₂)₈-S-(CH₂)₂-Ar |
| | | CH₃ | CH₃ | | |
| (41) | -(CH₂)₈-S-CH₂-Ar′ | -(CH₂)₄- | -(CH₂)₂- | -(CH₂)₈-S-CH₂-Ar′ | -(CH₂)₈-S-CH₂-Ar′ |
| | | CH₃ | CH₃ | | |
| (42) | -(CH₂)₈-S-CH(CH₃)- | -(CH₂)₄- | -(CH₂)₂- | -(CH₂)₈-S-CH(CH₃)- | -(CH₂)₈-S-CH(CH₃)- |
| | C(O)-OH | CH₃ | CH₃ | C(O)-OH | C(O)-OH |
| (43) | -(CH₂)₈-Hal | -(CH₂)₄- | -(CH₂)₂- | -(CH₂)₈-Hal | -(CH₂)₈-Hal |
| | | CH₃ | CH₃ | | |
| (44) | 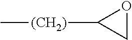 | -(CH₂)₄- | -(CH₂)₂- | 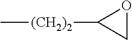 | 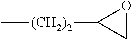 |
| | | CH₃ | CH₃ | | |

The PHA copolymer(s) of the invention preferably have a number-average molecular weight ranging from 50 000 to 150 000.

According to one embodiment, PHA copolymer(s) of the invention are preferably different from the compounds (7) and/or (7′), more preferably PHA copolymer(s) of the invention different from compound (7).

According to one embodiment preferably PHA copolymer(s) bears a R¹ of formula —(CH₂)ᵣ—X-(ALK)ᵤ-G wherein r, u and X are as defined previously, ALK represents a linear or branched, preferably linear, (C₁-C₈)alkylene.

The molecular weight may notably be measured by size exclusion chromatography. A method is described below in the examples.

The PHA copolymer(s) are particularly present in the composition according to the invention in a content ranging from 0.1% to 30% by weight and preferably ranging from 0.1% to 25% by weight relative to the total weight of the composition.

The PHA copolymer(s) preferably have a number-average molecular weight ranging from 50 000 to 150 000.

The molecular weight may notably be measured by size exclusion chromatography. A method is described below in the examples.

The copolymer may be present in the composition according to the invention in a content ranging from 0.1% to 30% by weight, and preferably from 0.1% to 25% by weight, relative to the total weight of the composition.

### Method for Preparing the PHA Copolymer(s):

The methods for preparing the PHA copolymer(s) of the invention are known to those skilled in the art. Mention may notably be made of the use of “functionalizable” PHA-producing microbial strains.

The term “functionalizable” means that the PHA copolymer(s) comprise a hydrocarbon-based chain comprising one or more atoms or groups that are capable of reacting chemically with another reagent—also referred to as “reactive atoms or reactive groups”- to give a Σ covalent bond functionalized by said reagent. The reagent is, for example, a compound comprising at least one nucleophilic group and said functionalized hydrocarbon-based chain comprises at least one electrophilic or nucleofugal atom or group, the nucleophilic group(s) reacting with the electrophilic group(s) to covalently graft Σ the reagent. The nucleophilic reagent may also react with one or more unsaturations of the alkenyl group(s) to also lead to grafting by covalent bonding of the functionalized hydrocarbon-based chain with said reagent. The addition reaction may also be radical-based, an addition of Markovnikov or anti-Markovnikov type, or nucleophilic or electrophilic substitution. The addition or condensation reactions may or may not take place via a radical route, with or without the use of catalysts or of enzymes, with heating preferably less than or equal to 100° C., under a pressure of greater than 1 atm, under an inert atmosphere or under oxygen.

The term “nucleophilic” refers to any atom or group which is electron-donating by an inductive effect+I and/or a mesomeric effect+M. Electron-donating groups that may be mentioned include hydroxyl, thiol and amino groups.

The term “electrophilic” refers to any atom or group which is electron-withdrawing by an inductive effect −I and/or a mesomeric effect −M. Electron-withdrawing species that may be mentioned include.

The microorganisms which produce PHAs of the invention notably bearing a hydrocarbon-based C₃-C₅ chain may be naturally produced by the bacterial kingdom, such as Cyanobacteria of the order of Nostocales (e.g.: Nostoc muscorum, Synechocystis and Synechococcus) but mainly by the Proteobacteria, for example in the class of:
beta-Proteobacteria, of the order Burkholderiales (Cupriavidus negator synonym Rasltonia eutropha)
alpha-Proteobacteria, of the order Rhodobacteriales (Rhodobacter capsulatus marine and photosynthetic)
gamma-Proteobacteria, of the order Pseudomonales of the family Moraxellaceae (Acinetobacter junii).

Among the microorganisms of the bacterial kingdom, the genera Azotobacter, Hydrogenomomas or Chromatium are the most representative of the PHA-producing organisms.

The organisms which naturally produce PHAs notably bearing a C₃-C₅ hydrocarbon-based chain are notably Proteobacteria, such as gamma-Proteobacteria, and more particularly of the order Pseudomonales of the family Pseudomonas such as Pseudomonas resinovorans, Pseudomonas putida, Pseudomonas fluorescens, Pseudomonas aeruginosa, Pseudomonas citronellolis, Pseudomonas mendocina, Pseudomonas chlororaphis and preferably Pseudomonas putida GPo1 and Pseudomonas putida KT2440, preferably Pseudomonas putida and Pseudomonas putida and in particular Pseudomonas putida GPo1 and Pseudomonas putida KT2440.

Certain organisms may also naturally produce PHAs without belonging to the order of Pseudomonales, such as Commamonas testosteroni which belongs to the class of beta-Proteobacteria of the order Burkholderiales of the family of Comamonadaceae.

The PHA-producing microorganism according to the invention may also be a recombinant strain if a 3-oxidation PHA synthase metabolic pathway is present. The 3-oxidation PHA synthase metabolic pathway is mainly represented by four classes of enzymes, EC: 2.3.1 B2, EC: 2.3.1 B3, EC: 2.3.1 B4 and EC: 2.3.1 B5.

The recombinant strain may be from the Bacteria kingdom, for example Escherichia coli or from the Plantae kingdom, for example Chlorella pyrenoidosa: International Journal of Biological Macromolecules, 116, 552-562 “Influence of nitrogen on growth, biomass composition, production, and properties of polyhydroxyalkanoates (PHAs) by microalgae”) or from the Fungi kingdom, for instance Saccaromyces cerevisiae or Yarrowia lipolytica: Applied Microbiology and Biotechnology 91, 1327-1340 (2011) “Engineering polyhydroxyalkanoate content and monomer composition in the oleaginous yeast Yarrowia lipolytica by modifying the β-oxidation multifunctional protein”).

Use may also be made of genetically modified microorganisms, which may make it possible, for example, to increase the production of PHA, and/or to increase the oxygen consumption capacity, and/or to reduce the autolysis and/or to modify the monomer ratio.

It is known that, for PHAs, a large portion of the total production cost is devoted to the culture medium and mainly to the substrate/carbon source. Use may thus be made of genetically modified microorganisms using a smaller amount of nutrient (carbon source) for their growth, for example microorganisms that are photo-autotrophic by nature, i.e. using light and CO₂ as main energy source.

The copolymer may be obtained in a known manner by biosynthesis, for example with the microorganisms belonging to the genus Pseudomonas, such as Pseudomonas resinovorans, Pseudomomonas putida, Pseudomonas fluorescens, Pseudomonas aeruginosa, Pseudomonas citronellolis, Pseudomonas mendocina, Pseudomonas chlororaphis and preferably Pseudomonas putida; and with a carbon source which may be a C₂-C₂₀, preferably C₆-C₁₈, carboxylic acid, such as acetic acid, propionic acid, butyric acid, hexanoic acid, heptanoic acid, octanoic acid, nonanoic acid, dodecanoic acid; a saccharide, such as fructose, maltose, lactose, xylose, arabinose, etc.); an n-alkane, such as hexane, octane or dodecane; an n-alcohol, such as methanol, ethanol, octanol or glycerol; methane or carbon dioxide.

The biosynthesis may optionally be performed in the presence of an inhibitor of the β-oxidation pathway, such as acrylic acid, methacrylic acid, propionic acid, cinnamic acid, salicylic acid, pentenoic acid, 2-butynoic acid, 2-octynoic acid or phenylpropionic acid, and preferably acrylic acid.

According to one embodiment, the process for preparing the PHAs of the invention uses microbial cells, which produce PHAs via genetically modified microorganisms (CMOs). The genetic modification may increase the production of PHA, increase the oxygen absorption capacity, increase the resistance to the toxicity of solvents, reduce the autolysis, modify the ratio of the PHA comonomers, and/or any combination thereof. In some of these embodiments, the modification of the comonomer ratio of the unit (A) increases the amount of predominant monomer versus (B) of the PHA of the invention, which is obtained. In another embodiment, the PHA-producing microbial cells reproduce naturally.

An example of genetically modified PHA-producing microbial strains is Pseudomonas entomophila LAC23 (Adv. Healthcare Mater. 2017, 1601017 (DOI: 10.1002/adhm.201601017.)

By way of example, a genetically modified microbial strain producing PHA that is functionalizable or comprising a reactive group that may be mentioned is Pseudomonas entomophila LAC23 (Biomacromolecules. 2014 Jun. 9; 15(6):2310-9. doi: 10.1021/bm500669s).

It is also possible to use wild-type strains which produce 100% PHO (according to the publication dx.doi.org/10.1021/bm2001999|Biomacromolecules 2011, 12, 2126-2136).

It is also possible to use genetically modified microorganisms which produce phenylvaleric-co-3-hydroxydodecanoic copolymers HDD (Sci. China Life Sci., Shen R., et al., 57 No. 1, (2014) with a strain: Pseudomonas entomophila LAC23.

Nutrients, such as water-soluble salts based on nitrogen, phosphorus, sulfur, magnesium, sodium, potassium and iron, may also be used for the biosynthesis.

The known appropriate temperature, pH and dissolved oxygen (O_{D}) conditions may be used for the culturing of the microorganisms.

The microorganisms may be cultured according to any known method of culturing, such as in a bioreactor in continuous or batch mode, in fed or unfed mode.

The biosynthesis of the polymers used according to the invention is notably described in the article “Biosynthesis and Properties of Medium-Chain-Length Polyhydroxyalkanoates with Enriched Content of the Dominant Monomer”, Xun Juan et al., Biomacromolecules 2012, 13, 2926-2932, and in patent application WO 2011/069244.

The microbial strains producing PHA which is functionalizable or comprising a reactive group, as defined previously, are, for example, of the genus Pseudomonas such as P. cichorii YN2, P. citronellolis, P. jessenii, and more generally with species of Pseudomonas putida such as Pseudomonas putida GPo1 (synonym of Pseudomonas oleovorans), P. putida KT2442, P. putida KCTC 2407, P. putida BM01.

The Carbon Source(s):

One means for gaining access to the PHAs of the invention is to introduce one or more organic compounds into the culture medium, this or these organic compounds representing a carbon source preferably chosen from alkanes, alkenes, alcohols, carboxylic acids and a mixture thereof.

In one embodiment, the organic compound(s) will preferably be chosen from alcohols, carboxylic acids and a mixture thereof.

The carbon source(s) may be classified as follows:

1) Carbon Source Via One or More Organic Compounds Introduced into the Medium:

One means for gaining access to the PHAs of the invention is to introduce one or more organic compounds into the culture medium, this organic compound being a carbon source preferably chosen from alkanes, alkenes, alcohols, carboxylic acids and mixtures thereof.

According to a particular embodiment of the invention, the organic compound(s) are chosen from alcohols, in particular (C₅-C₂₀)alkanols, and/or carboxylic acids, in particular (C₅-C₂₀)alkanoic acids.

The carbon source(s) may be classified into three groups:
group A: the organic compound may aid the growth of the productive strain and aid the production of PHA structural linked to the organic compound.
group B: the organic compound may aid the growth of the strain but does not participate in the production of PHA structural linked to the organic compound.
group C: the organic compound does not participate in the growth of the strain.

Such microbiological processes are known to those skilled in the art, notably in the scientific literature. Mention may be made of: International Journal of Biological Macromolecules 28, 23-29 (2000); The Journal of Microbiology, 45, No. 2, 87-97, (2007).

According to one variant, the integration of the substrate that is structurally linked to the reactive atom(s) or to the reactive group(s) of the PHAs of the invention is introduced directly into the medium as sole carbon source in a medium suitable for microbial growth. (Example: group A for P. putida GPo1: alkenoic acid, notably terminal).

According to another variant, the integration of the substrate that is structurally linked to the reactive atom(s), notably halogen, or to the reactive group(s) of the PHAs of the invention is introduced into the medium as carbon source with a second carbon source as co-substrate which is also structurally linked to the PHA, in a medium suitable for microbial growth. (Example: group B for P. putida GPo1: haloalkanoic acids which are preferably terminal, such as terminal bromoalkanoic acids).

According to yet another variant, the integration of the substrate that is structurally linked to the reactive atom(s), notably halogen, or to the reactive group(s) of the PHAs of the invention may be introduced directly into the medium as carbon source with a second carbon source as co-substrate which is also structurally linked to the PHAs and a third carbon source as co-substrate which is not structurally linked to the PHAs, in a medium suitable for microbial growth. (Example: group C glucose or sucrose).

In one embodiment, the β-oxidation pathway inhibitor is acrylic acid, 2-butynoic acid, 2-octynoic acid, phenylpropionic acid, propionic acid, trans-cinnamic acid, salicylic acid, methacrylic acid, 4-pentenoic acid or 3-mercaptopropionic acid.

In one embodiment of the first aspect, the functionalized fatty acid is a functionalized hexanoic acid, functionalized heptanoic acid, functionalized octanoic acid, functionalized nonanoic acid, functionalized decanoic acid, functionalized undecanoic acid, functionalized dodecanoic acid or functionalized tetradecanoic acid.

The functionalization may be introduced by means of an organic compound chosen from precursors of the alcohol and/or carboxylic acid category, notably:
for functionalization of the PHA(s) with a branched alkyl group: see, for example Applied and Environmental Microbiology, 60, No. 9, 3245-325 (1994);
for functionalization of the PHA(s) with a linear alkyl group comprising a terminal cyclohexyl unit: see, for example doi.org/10.1016/S0141-8130(01)00144-1;
for functionalization of the PHA(s) with an unsaturated alkyl group which is preferably terminal: see, for example doi.org/10.1021/bm8005616);
for functionalization of the PHA(s) with a linear alkyl group comprising a halogen preferably at the end of the hydrocarbon-based chain (doi.org/10.1021/ma00033a002);
for functionalization of the PHA(s) with a (hetero)aromatic alkyl group, for example phenyl, benzoyl, phenoxy, see, for example J. Microbiol. Biotechnol., 11, 3, 435-442 (2001);
for functionalization of the PHA(s) with a linear alkyl group comprising a heteroatom notably at the end of the hydrocarbon-based chain, see, for example DOI 10.1007/s00253-011-3099-4;
for functionalization of the PHA(s) with a linear alkyl group comprising a cyano function notably at the end of the hydrocarbon-based chain, see, for example doi.org/10.1111/j.574-6968.1992.tb05839.x;
for functionalization of the PHA(s) with a linear alkyl group comprising an epoxy function notably at the end of the hydrocarbon-based chain, see, for example doi.org/10.1016/S1381-5148(97)00024-2;
   The review International Microbiology 16:1-15 (2013) doi:10.2436/20.1501.01.175) also mentions the majority of the functionalized native PHAs.

In a particular embodiment of the invention, the fatty acid from group A is chosen from 11-undecenoic acid, 10-epoxyundecanoic acid, 5-phenylvaleric acid, citronellol and 5-cyanopentanoic acid.

In a particular embodiment of the invention, the fatty acid from group B is chosen from halooctanoic acids such as 8-bromooctanoic acid and 11-bromoundecanoic acid.

In a particular embodiment of the invention, the carbon source from group C is a monosaccharide, preferably glucose.

2) Carbon Source in the Presence of Oxidation Inhibitor Introduced into the Medium:

Another aspect of the invention is the use of the PHA-producing microbial strains in a medium that is suitable for microbial growth, said medium comprising: a substrate which is structurally linked to the PHA(s); at least one carbon source which is not structurally linked to the PHA(s); and at least one oxidation and notably β-oxidation pathway inhibitor. This allows the growth of the microbial cells to take place in said medium, the microbial cells synthesizing the PHA polymer(s) of the invention; preferably copolymer particularly containing more than 95% of identical units, which has a comonomer ratio of unit (A) and of unit (B) which differs from that obtained in the absence of the β-oxidation pathway inhibitor.

Example of functionalization of PHA copolymers according to the invention starting from a) a PHA copolymer bearing an unsaturated hydrocarbon-based chain, according to Scheme 1 below:

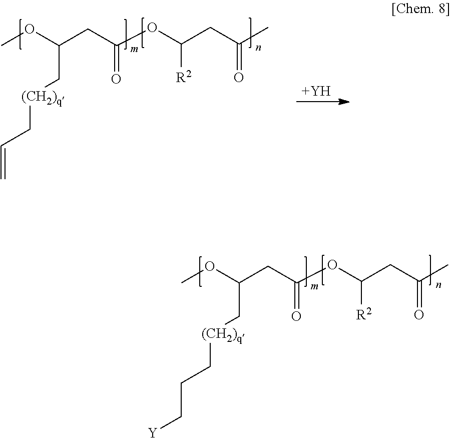

in which Scheme 1:
R², m and n are as defined previously;
Y represents a group chosen from Hal such as chlorine or bromine, hydroxyl, thiol, (di)(C₁-C₄)(alkyl)amino, R—X with R representing a group chosen from α) cycloalkyl such as cyclohexyl, β) heterocycloalkyl such as a sugar, preferably a monosaccharide such as glucose, γ) (hetero)aryl such as phenyl; δ) a cosmetic active agent as defined previously; ε) (C₁-C₂₀)alkyl, (C₂-C₂₀)alkenyl, (C₂-C₂₀)alkynyl; and X representing a′) O, S, N(Rₐ) or Si(R_{b})(R_{c}) or e) linear or branched (C₁-C₂₀)alkyl, with Rₐ, R_{b} and R_{c} as defined previously;
q′ represents an integer inclusively between 2 and 20, preferably between 3 and 10, more preferentially between 4 and 8 such as 6, better still between 3 and 8, preferably between 4 and 6, such as 5.

Other reactions may be performed using double or triple unsaturations such as Michael or Diels-Alder addition, radical addition, catalytic (notably with Pd or Ni) or non-catalytic hydrogenation, halogenation, notably with bromine, hydration or oxidation, which may or may not be controlled, and reaction on electrophiles as represented schematically below.

According to a particular embodiment of the invention, the PHA copolymers comprise a linear or branched, saturated hydrocarbon-based chain R¹, substituted and/or interrupted with groups as defined previously for R¹, comprising in total between 5 and 30 carbon atoms, preferably between 6 and 20 carbon atoms and more particularly between 7 and 11 carbon atoms, and a hydrocarbon-based chain of R² represents a linear or branched (C₃-C₂₀)alkenyl, particularly (C₅-C₁₄)alkenyl and more particularly (C₇-C₁₀)alkenyl radical, which is preferably linear and comprising only one unsaturation at the chain end, in particular —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5 comprising between 1% and 99%, preferentially between 2% and 50% and even more preferentially between 3% and 40% of unsaturations, and even more particularly between 3% and 30% of unsaturations, better still between 5% and 20% of unsaturations.

A) Copolymer PHA with unsaturations may be chemically modified:

via addition reactions, such as radical additions, Michael additions, electrophilic additions, Diels-Alder, halogenation, hydration or hydrogenation reaction, and preferably hydrothiolation reaction with particles, chemical compounds or polymers. In particular, the hydrothiolation reactions may be performed in the presence of a thermal initiator, a redox initiator or a photochemical initiator and of an organic compound bearing a sulfhydryl group, notably chosen from:
linear, branched, cyclic or aromatic alkanethiols including 1 to 14 carbon atoms, such as methane-, ethane-, propane-, pentane-, cyclopentane-thiol, preferably hexane-, cyclohexane-, heptane-, octane-, phenylethane-, 4-tert-butylphenylmethane- or 2-furanmethane-thiol;
organosiloxane bearing a thiol function, such as (3-mercaptopropyl)trimethoxysilane, (3-mercaptopropyl) methyldimethoxysilane, 2-(triethoxysilyl)ethanethiol or mercaptopropyl-isobutyl-POSS;
thiol-based silicone oils, notably those described in the document DOI: 10.1016/j.actbio.2015.01.020);
thiol-based oligomers or polymers bearing a reactive function, such as an amine, an alcohol, an acid, a halogen, a thiol, an epoxide, a nitrile, an isocyanate, a heteroatom, preferably cysteine, cysteamine, N-acetylcysteamine, 2-mercaptoethanol, 1-mercapto-2-propanol, 8-mercapto-1-octanol, thiolactic acid, thioglycolic acid, 3-mercaptopropionic acid, 11-mercaptoundecanoic acid, polyethylene glycol dithiol, 3-mercaptopropionitrile, 1,3-propanedithiol, 4-cyano-1-butanethiol, 3-chloro-1-propanethiol, 1-thio-β-D-glucose tetraacetate; and
thiols which may be obtained from disulfide reduction, such as phenyl disulfide or furfuryl disulfide.

Examples of initiators that may be mentioned include: tert-butyl peroxy-2-ethylhexanoate, cumene perpivalate, tert-butyl peroxylaurate, benzoyl peroxide, lauroyl peroxide, octanoyl peroxide, di-tert-butyl peroxide, tert-butylcumyl peroxide, dicumyl peroxide, 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1-bis(tert-butylperoxy)-3,3,5-trimethylcyclohexane, 1,1-bis(tert-butylperoxy)cyclohexane, 1,4-bis(tert-butylperoxycarbonyl)cyclohexane, 2,2-bis(tert-butylperoxy)octane, n-butyl 4,4-bis(tert-butylperoxy)valerate, 2,2-bis(tert-butylperoxy)butane, 1,3-bis(tert-butylperoxyisopropyl)benzene, 2,5-dimethyl-2,5-bis(tert-butylperoxy)hexane, 2,5-dimethyl-2,5-di(benzoylperoxy)hexane, di-tert-butyl diperoxyisophthalate, 2,2-bis(4,4-di-tert-butylperoxycyclohexyl)propane, di-tert-butyl peroxy-α-methylsuccinate, di-tert-butyl peroxydimethylglutarate, di-tert-butyl peroxyhexahydroterephthalate, di-tert-butyl peroxyazelate, 2,5-dimethyl-2,5-bis(tert-butylperoxy)hexane, diethylene glycol bis(tert-butylperoxycarbonate), di-tert-butyl peroxytrimethyladipate, tris(tert-butylperoxy)triazine, vinyltris(tert-butylperoxy)silane phenothiazine, tetracene, perylene, anthracene, 9,10-diphenylanthracene, thioxanthone, benzophenone, acetophenone, xanthone, fluorenone, anthraquinone, 9,10-dimethylanthracene, 2-ethyl-9,10-dimethyloxyanthracene, 2,6-dimethylnaphthalene, 2,5-diphenyl-1,3,4-oxadiazole, xanthopinacol, 1,2-benzanthracene, 9-nitroanthracene. Each of these initiators may be used alone or in combination of others.
The chemical reactions mentioned previously are known to those skilled in the art. Mention may notably be made of the following documents: Synthesis and preparation of PHAs modified with polyethylene glycol dithiol: 10.1021/acs.biomac.9b00479; Biomacromolecules, 19, 3536-3548 (2018);
Synthesis and preparation of PHAs modified with mercaptohexanol: 10.1021/acs.biomac.8b01257; Biomacromolecules, 20, 2, 645-652 (2019);
Synthesis and preparation of PHAs modified with hydroxycinnamic acid sulfate, and zosteric acid: 10.1021/bm049962e; Biomacromolecules, 5, 4, 1452-1456 (2004);
Radical addition of methyl methacrylate to a PHOUn: 10.1002/1521-3935(20010701)202:11<2281::AID-MACP2281>3.0.CO; 2-9; Macromolecular Chemistry and Physics, vol. 202, 11, 2281-2286 (2001); Synthesis and preparation of PHAs modified with a polysilsesquioxane (POSS): 10.1016/j.polymer.2005.04.020; Polymer Vol. 46, 14, 5025-5031 (2005);
Grafting of thio-beta-glucose to saturated side chains: 1022-1336/99/0202-0091$17.50+0.50/0; Macromol. Rapid Commun., 20, 91-94 (1999)

B) Copolymer PHA with unsaturations may also be chemically modified via oxidation reactions, which may or may not be controlled, for example with the permanganates of a concentrated or dilute alkaline agent, or ozonolysis, oxidation in the presence of a reducing agent, making it possible to obtain novel materials bearing hydroxyl, epoxide or carboxyl groups in the terminal position of the side chains:

Scheme 2: a few examples of chemical modifications via oxidation of PHA bearing an unsaturation in the terminal position of the side chain

The chemical reactions mentioned previously are known to those skilled in the art. Mention may notably be made of the following documents: 10.1021/bm049337; Biomacromolecules, vol. 6, 2, 891-896 (2005); 10.1016/S0032-3861(99)00347-X; Polymer, vol. 41, 5, 1703-1709 (2000); 10.1021/ma9714528 and 10.1016/S1381-5148(97)00024-2; Macromolecules, 23, 15, 3705-3707 (1990); 10.1016/S0032-3861(01)00692-9; Polymer, vol. 43, 4, 1095-1101 (2002); 10.1016/S0032-3861(99)00347-X; Polymer, vol. 41, 5, 1703-1709 (2000); and 10.1021/bm025728h; Biomacromolecules, vol. 4, 2, 193-195 (2003).

Example of functionalization of PHA copolymers according to the invention starting from b) a PHA copolymer bearing a hydrocarbon-based chain containing an epoxide group, according to Scheme 2 below:

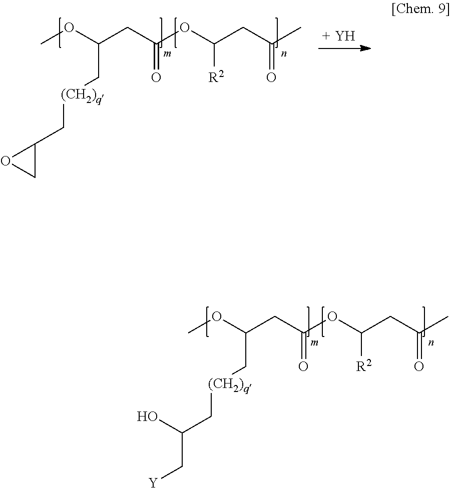

in which Scheme 2 Y, m, n, q′ and R2 are as defined in Scheme 1.

The epoxide structure may be obtained via a conventional method known to those skilled in the art, whether via biotechnological processes or via chemical processes such as oxidation of unsaturation as mentioned previously. The peroxide group(s) may react with carboxylic acids, maleic anhydrides, amines, alcohols, thiols or isocyanates, all these reagents including at least one linear or branched, cyclic or acyclic, saturated or unsaturated C₁-C₂₀ hydrocarbon-based chain, or borne by an oligomer or polymer, in particular amino (poly)saccharides such as compounds derived from chitosan and (poly)sil(ox)anes; 3-glycidyloxypropyltrimethoxysilane, 3-aminopropyltriethoxysilane 3-(trimethoxysilyl)propylcarbamic acid, diethanolamine, or 3-mercapto-1-propanesulfonate of alkali metal or alkaline-earth metal salts such as sodium. The epoxide groups may also react with water.

Mention may notably be made of the following documents:
Preparation of PHA bearing charges starting with diethanolamine: 10.1021/bm8005616, Biomacromolecules, vol. 9, 8 2091-2096 (2008);
Preparation of PHA bearing charges starting with sodium 3-mercapto-1-propanesulfonate: 10.1021/acs.biomac.9b00870 Biomacromolecules, vol. 20, 9, 3324-3332 (2019);
Preparation of PHA including a native epoxide unit: 10.1016/S1381-5148(97)00024-2); Reactive and Functional Polymers, vol. 34, 1, 65-77 (1997)

Example of functionalization of PHA copolymers according to the invention starting from c) a PHA copolymer bearing a hydrocarbon-based chain containing a nucleofugal group, according to Scheme 3 below:

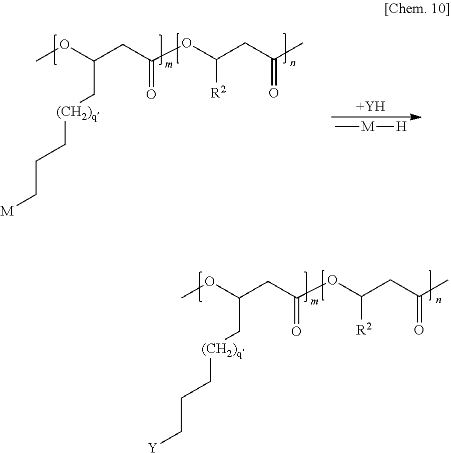

in which Scheme 3 Y, m, n, q′ and R² are as defined in Scheme 1. M corresponds to an organic or inorganic nucleofugal group, which may be substituted with a nucleophilic group; preferably, said nucleophile is a heteroatom which is electron-donating via the +I and/or +M effect such as O, S or N. Preferably, the nucleofugal group M is chosen from halogen atoms such as Br, and mesylate, tosylate or triflate groups. This is a reaction known to those skilled in the art. Mention may be made, for example, of the following document: 10.1016/j.ijbiomac.2016.11.118, International Journal of Biological Macromolecules, vol. 95, 796-808 (2017).

Example of functionalization of PHA copolymers according to the invention starting from d) a PHA copolymer bearing a hydrocarbon-based chain containing a cyano group, according to Scheme 4 below:

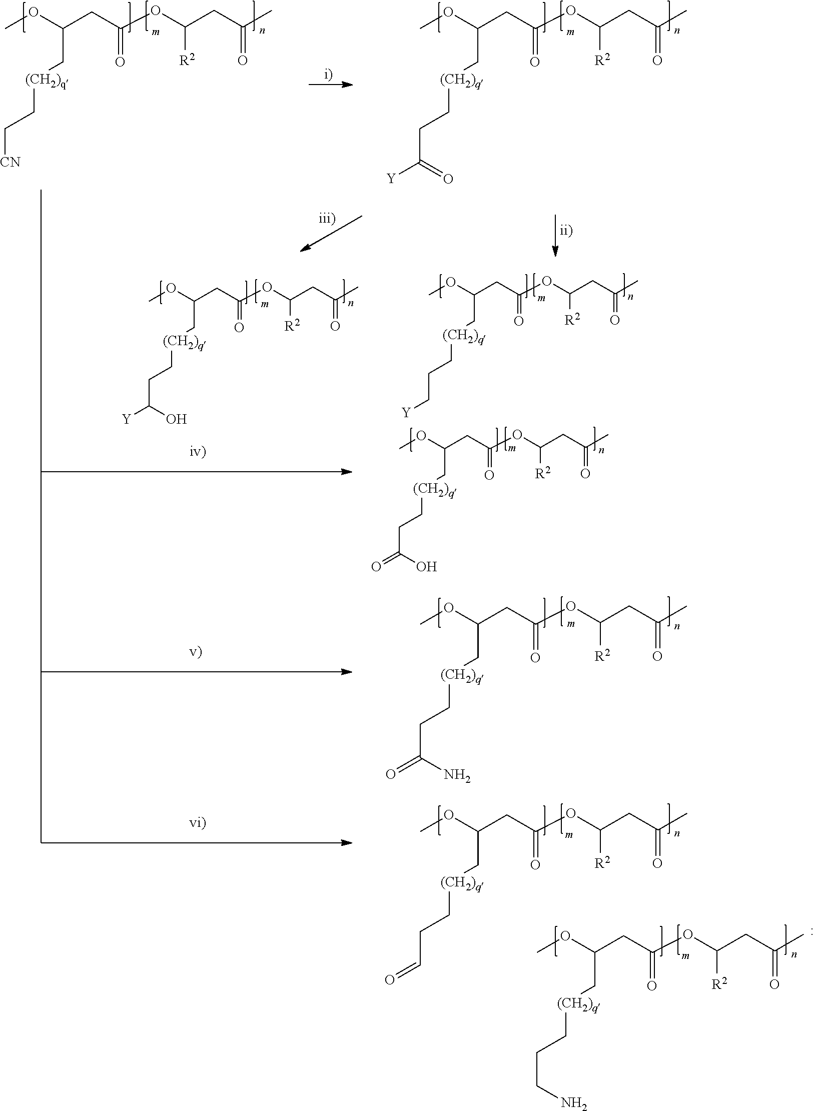

in which Scheme 4 Y, m, n, q′ and R₂ are as defined in Scheme 1.

In a first step i), the PHA copolymer bearing a side chain containing a cyano or nitrile group reacts with an organo-alkali metal or organomagnesium compound Y-MgHal, Y—Li or Y—Na, followed by hydrolysis to give the PHA copolymer bearing a side chain containing a group Y grafted with a ketone function. The ketone function may be converted into a thio ketone by thionation, for example with S8 in the presence of amine, or with Lawesson's reagent. Said thio ketone, after total reduction ii) (for example by Clemmensen reduction) leads to the PHA copolymer bearing a side chain containing a group Y grafted with an alkylene group. Alternatively, said thio ketone may undergo a controlled reduction iii) with a conventional reducing agent to give the PHA copolymer bearing a side chain containing a group Y grafted with a hydroxyalkylene group. The cyano group of the starting PHA copolymer can react with water after hydration v) to give the amide derivative, or after hydrolysis iv) to the carboxyl derivative. The cyano group of the starting PHA copolymer may also, after reduction vi), give the amine derivative or the ketone derivative. The PHA copolymers bearing a hydrocarbon-based side chain containing a nitrile function are prepared via conventional methods known to those skilled in the art. Mention may be made, for example, of the document: 10.1016/0378-1097(92)90311-B, FEMS Microbiology Letters, vol. 103, 2-4, 207-214 (1992).

Example of functionalization of PHA copolymers according to the invention starting from e) a PHA copolymer bearing a hydrocarbon-based chain at the chain end, according to Scheme 5 below:

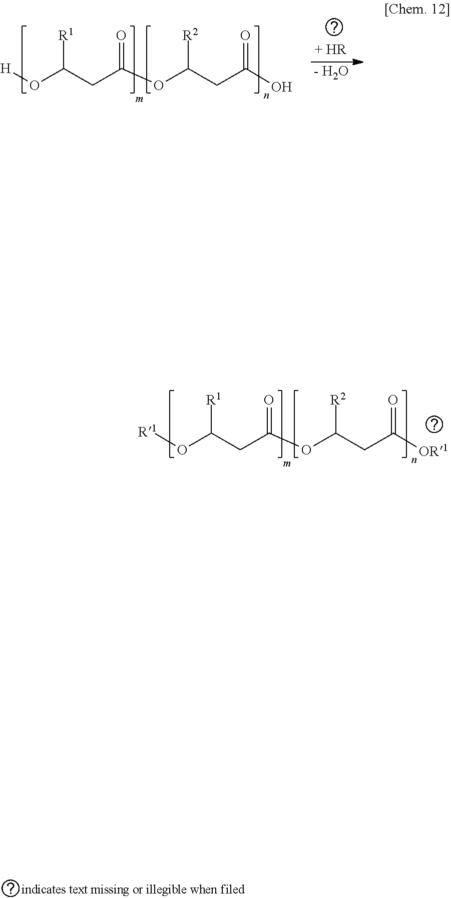

in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; preferably, the hydrocarbon-based group is linear; said hydrocarbon-based chain being substituted with one or more atoms or groups chosen from: a) halogens such as chlorine or bromine, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl such as phenyl or furyl, and i) (hetero)cycloalkyl such as anhydride, or epoxide, j) a cosmetic active agent chosen from coloured or uncoloured, fluorescent or non-fluorescent chromophores such as those derived from optical brighteners, or chromophores derived from UVA and/or UVB screening agents, and anti-ageing active agents.

These chain-end grafting agents on PHA polymers are known to those skilled in the art. Mention may be made, for example, of the following documents:
Preparation of PHA oligomers by thermal degradation: 10.1021/bm0156274; Biomacromolecules, vol. 3, 1, 219-224 (2002);
Preparation of PHA oligomers by transesterification: 10.1021/ma011420r, Macromolecules, vol. 35, 3, 684-689 (2002);
Preparation of PHA oligomers by hydrolysis: 10.1016/0032-3861(94)90590-8 Polymer vol. 35, 19, 4156-4162 (1994);
Preparation of PHA oligomers by methanolysis: 10.1021/bm060981t, Biomacromolecules, vol. 8, 4, 1255-1265 (2007).

Mention may also be made of other methods known to those skilled in the art:
Synthesis and characterization of PHA grafted with ascorbic acid: 10.1016/j.ijbiomac.2018.11.052; International Journal of Biological Macromolecules, vol. 123: 7 (2019);
Preparation of PHB-b-PHO copolymers by polycondensation with divinyl adipate catalysed with a lipase: 10.1021/bm9011634, Biomacromolecules, vol. 10, 12, 3176-3181 (2009);
Synthesis of PHB-b-PHO copolymers coupled via a diisocyanate junction: 10.1021/ma012223v; Macromolecules, vol. 35, 13, 4946-4950 (2002);
Preparation of PHO oligomers on chitosan by condensation between the carboxylic acid end of the PHO and the amine functions of the chitosan: 10.1002/app.24276; Journal of Applied Polymer Science, vol. 103, 1, (2006);
Transesterification of PHAs with propargyl alcohol in order to produce PHA oligomers that are modifiable by “click” chemistry: 10.1016/j.reactfunctpolym.2011.12.005; Reactive and Functional Polymers, vol. 72, 2, 160-167 (2012);
Preparation of PHO-b-PCL copolymer: 10.1002/mabi.200400104; Macromolecular Bioscience, vol. 4, 11 (2004);
Preparation of PHO-b-PEG copolymer: 10.1002/macp.201000562; Macromolecular Chemistry and Physics; vol. 212, 3, (2010);
Epoxidation of chain-end unsaturation and chain-end grafting of acid: 10.14314/polimery.2017.317; Polimery, vol. 62, 4, 317-322 (2017);
Grafting of organosiloxane unit at chain end onto PHA: 10.1016/j.reactfunctpolym.2014.09.008; Reactive and Functional Polymers, vol. 84, 53-59 (2014).

The combination of grafted PHA copolymers of the invention described previously, starting from f) a PHA bearing a reactive atom or group, according to Scheme 6:

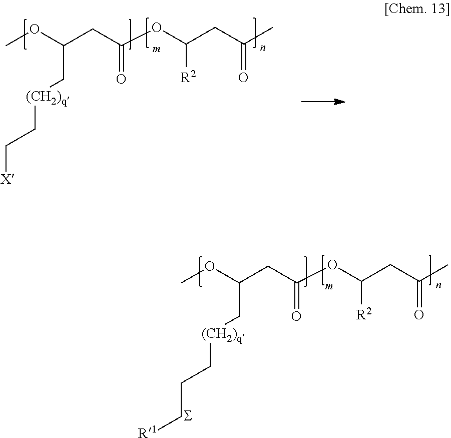

in which Scheme 6 R′¹, R², m, n and Y are as defined previously, and X′ represents a reactive atom or group that is capable of reacting with an electrophilic [symbol] or nucleophilic [symbol] atom or group to create a Σ covalent bond; if X′ is an electrophilic or nucleofugal group, then it can react with a reagent R′¹- [symbol] ; if X′ is a nucleophilic group [symbol] , then it can react with R′¹- [symbol] to create a Σ covalent bond;

By way of example, the covalent bonds or bonding group that may be generated are listed in the table below, from condensation of electrophiles with nucleophiles:

TABLE 1

| Electrophiles [symbol] | Nucleophiles [symbol] u | Covalent bonds |
|---|---|---|
| Activated esters* | Amines | Carboxamides |
| Acyl azides** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salt thereof | Thiols | Thioethers |
| Sulfonic acids and salt thereof | Carboxylic acids | Esters |
| Sulfonic acids and salt thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Aziridines | Thiols | Thiolethers |
| Carboxylic acids | Amines | Carboxamides |
| Carboxylic acids | Alcohols | Esters |
| Carbodiimides | Carboxylic acids | N-acylureas |
| Diazolakanes | Carboxylic adds | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*the activated esters of general formula -CO-LG, with LG representing a leaving group such as oxysuccinimidyl, oxybenzotriazolyl or aryloxy, optionally substituted;
**the acyl azides can rearrange to give isocyanate.

It is also possible, starting with g) a PHA functionalized on a side chain, to perform chain-end grafting in a second stage as described in Scheme 7. The reciprocal is also true, in which the chain-end grafting may be performed in a first stage, followed by performing functionalization of a functionalizable side chain in a second stage.

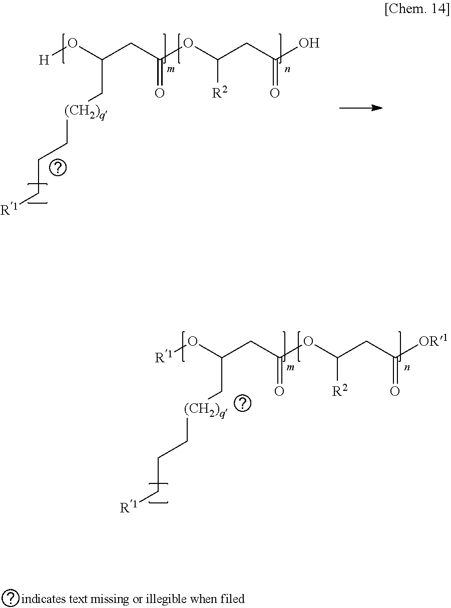

in which Scheme 7 R′¹, R², m, n and Y are as defined previously, and

All these chemical reactions are known to those skilled in the art. Mention may be made, for example, of the following documents:
Synthesis and preparation of PHAs modified with thiol-ene followed by reaction on the new grafted function: 10.1021/ma0304426; Macromolecules, vol. 37, 2, 385-389 (2004);
Grafting of PEG and of PLA onto PHAs functionalized with acids: 10.1002/marc.200900803 and 10.1002/mabi.200390033;
Synthesis and preparation of PHAs modified with polyethylene glycol dithiol: 10.1021/acs.biomac.9b00479.

### b) The Fatty Medium

The composition comprises as second ingredient a preferably oily fatty medium.

The composition may also comprise water. Preferably, the composition of the invention predominantly comprises on a weight basis one or more fatty substances versus the amount by weight of water.

The term “fatty substance” means an organic compound that is insoluble in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

The fatty substance(s) of the invention are of natural or synthetic origin, preferably natural, more preferentially of plant origin. These fatty substances are preferably neither polyoxyethylenated nor polyglycerolated. They are different from fatty acids since salified fatty acids constitute soaps which are generally soluble in aqueous media.

According to a particular embodiment of the invention, the composition comprises one or more fatty substances that are not liquid at 25° C. and at atmospheric pressure.

### The Wax(es)

According to a particular embodiment, the composition of the invention comprises one or more waxes.

The term “wax” means a lipophilic compound that is solid at room temperature (25° C.), with a reversible solid/liquid change of state, having a melting point of greater than or equal to 30° C., which may be up to 200° C. and notably up to 120° C.

In particular, the wax(es) that are suitable for use in the invention may have a melting point of greater than or equal to 45° C. and in particular of greater than or equal to 55° C.

The composition according to the invention preferably comprises a content of wax(es) ranging from 3% to 20% by weight relative to the total weight of the composition, in particular from 5% to 15% and more particularly from 6% to 15%.

According to a particular form of the invention, the composition of the invention is solid, in particular anhydrous. It may then be in stick form; use will be made of polyethylene microwaxes in the form of crystallites with an aspect ratio at least equal to 2, and with a melting point ranging from 70 to 110° C. and preferably from 70 to 100° C., so as to reduce or even eliminate the presence of strata in the solid composition. These crystallites in needle form and notably the dimensions thereof may be characterized visually according to the following method.

### The Pasty Compound(s)

According to a particular embodiment, the composition of the invention comprises one or more pasty compounds.

For the purposes of the present invention, the term “pasty compound” means a lipophilic fatty compound that undergoes a reversible solid/liquid change of state, having anisotropic crystal organization in the solid state, and including, at a temperature of 23° C., a liquid fraction and a solid fraction.

Preferably, the fatty medium comprises one or more liquid fatty substances; in particular, the liquid fatty substance(s) are chosen from non-silicone oils; preferably, the liquid fatty substance(s) are chosen from:
ester oils, carbonate oils;
apolar branched hydrocarbon-based oils containing from 8 to 14 carbon atoms, as a mixture with a monoalcohol containing from 2 to 6 carbon atoms in a monoalcohol/apolar branched hydrocarbon-based oil weight ratio preferably ranging from 1/99 to 10/90

### The Oil(s)

Preferably, a composition comprises one or more oils.

The term “oil” means a hydrophobic (i.e. water-immiscible) fatty (i.e. non-aqueous) substance that is liquid at room temperature (25° C.) and at atmospheric pressure (1 atm or 760 mmHg).

The term “liquid fatty substances” notably means liquid fatty substance(s) preferably having a viscosity of less than or equal to 7000 centipoises at 20° C.

The liquid fatty substance(s) of the invention more particularly have a viscosity of less than or equal to 2 Pa·s, more particularly less than or equal to 1 Pa·s, even more particularly less than or equal to 0.1 Pa·s, and more preferentially less than or equal to 0.09 Pa·s at a temperature of 25° C. and at a shear rate of 1 s⁻¹.

According to a particular embodiment of the invention, the liquid fatty substance(s) have a viscosity of between 0.001 Pa·s and 2 Pa·s, more particularly inclusively between 0.01 and 1 Pa·s and even more particularly inclusively between 0.014 and 0.1 Pa·s, more preferentially inclusively between 0.015 and 0.09 Pa·s at a temperature of 25° C. and at a shear rate of 1 s⁻¹.

The PHA copolymer(s) according to the invention are soluble in the liquid fatty substances at 25° C. and at atmospheric pressure.

According to the invention, the medium is said to be carbon-based if it comprises at least 50% by weight, notably from 50% to 100% by weight, for example from 60% to 99% by weight, or else from 65% to 95% by weight, or even from 70% to 90% by weight, relative to the total weight of the carbon-based medium, of carbon-based compound, which is liquid at 25° C.

Preferably, the liquid fatty substance(s) have an overall solubility parameter according to the Hansen solubility space of less than or equal to 20 (MPa)^{1/2}, or a mixture of such compounds.

The global solubility parameter δ according to the Hansen solubility space is defined in the article “Solubility parameter values” by Grulke in the book “Polymer Handbook”, 3rd Edition, Chapter VII, pages 519-559, by the relationship δ=(dD₂+dP₂+dH₂)^{1/2} in which:
d_{D} characterizes the London dispersion forces derived from the formation of dipoles induced during molecular impacts,
dₚ characterizes the Debye interaction forces between permanent dipoles,
d_{H} characterizes the forces of specific interactions (such as hydrogen bonding, acid/base, donor/acceptor, etc.).

The definition of solvents in the Hansen three-dimensional solubility space is described in the article by Hansen: “The three-dimensional solubility parameters”, J. Paint Technol. 39, 105 (1967).

Among the liquid carbon-based compounds having an overall solubility parameter according to the Hansen solubility space of less than or equal to 20 (MPa)^{1/2}, mention may be made of liquid fatty substances, notably oils, which may be chosen from natural or synthetic, carbon-based, or hydrocarbon-based oils, which are optionally fluorinated, and optionally branched, alone or as a mixture.

The liquid fatty substances are notably chosen from C₆-C₁₆ hydrocarbons or hydrocarbons comprising more than 16 carbon atoms and up to 60 carbon atoms and in particular alkanes, oils of animal origin, oils of plant origin, glycerides or fluoro oils of synthetic origin, fatty alcohols, fatty acid and/or fatty alcohol esters, non-silicone waxes, and silicones.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, dodecane and isoparaffins such as isohexadecane and isodecane. The linear or branched hydrocarbons containing more than 16 carbon atoms may be chosen from liquid paraffins, petroleum jelly, liquid petroleum jelly, polydecenes, and hydrogenated polyisobutene.

According to a particular embodiment, the fatty substance(s) used in the process of the invention are chosen from volatile linear alkanes.

The term “one or more volatile linear alkanes” means, without distinction, “one or more volatile linear alkane oils”.

A volatile linear alkane that is suitable for use in the invention is liquid at room temperature (about 25° C.) and atmospheric pressure (101 325 Pa or 760 mmHg).

The term “volatile linear alkane” that is suitable for use in the invention means a linear alkane that can evaporate on contact with the skin in less than one hour, at room temperature (25° C.) and atmospheric pressure (101 325 Pa), which is liquid at room temperature, notably having an evaporation rate ranging from 0.01 to 15 mg/cm²/minute, at room temperature (25° C.) and atmospheric pressure (101 325 Pa).

Preferably, the volatile linear alkanes that are suitable for use in the invention have an evaporation rate ranging from 0.01 to 3.5 mg/cm²/minute and better still from 0.01 to 1.5 mg/cm²/minute, at room temperature (25° C.) and atmospheric pressure (101 325 Pa).

More preferably, the volatile linear alkanes that are suitable for use in the invention have an evaporation rate ranging from 0.01 to 0.8 mg/cm²/minute, preferentially from 0.01 to 0.3 mg/cm²/minute and even more preferentially from 0.01 to 0.12 mg/cm²/minute, at room temperature (25° C.) and atmospheric pressure (101 325 Pa).

The evaporation rate of a volatile alkane in accordance with the invention (and more generally of a volatile solvent) may notably be evaluated by means of the protocol described in WO 06/013 413, and more particularly by means of the protocol described below.

15 g of volatile hydrocarbon-based solvent are placed in a crystallizing dish (diameter: 7 cm) placed on a balance that is in a chamber of about 0.3 m³ with regulated temperature (25° C.) and hygrometry (50% relative humidity).

The volatile hydrocarbon-based solvent is allowed to evaporate freely, without stirring it, while providing ventilation by means of a fan (Papst-Motoren, reference 8550 N, rotating at 2700 rpm) placed in a vertical position above the crystallizing dish containing the volatile hydrocarbon-based solvent, the blades being directed towards the crystallizing dish, 20 cm away from the bottom of the crystallizing dish.

The mass of volatile hydrocarbon-based solvent remaining in the crystallizing dish is measured at regular time intervals.

The evaporation profile of the solvent is then obtained by plotting the curve of the amount of product evaporated (in mg/cm²) as a function of the time (in min).

The evaporation rate is then calculated, which corresponds to the tangent to the origin of the curve obtained. The evaporation rates are expressed in mg of volatile solvent evaporated per unit area (cm²) and per unit time (minutes).

According to a preferred embodiment, the volatile linear alkanes that are suitable for use in the invention have a non-zero vapour pressure (also known as the saturation vapour pressure), at room temperature, in particular a vapour pressure ranging from 0.3 Pa to 6000 Pa.

Preferably, the volatile linear alkanes that are suitable for use in the invention have a vapour pressure ranging from 0.3 to 2000 Pa and better still from 0.3 to 1000 Pa, at room temperature (25° C.).

More preferably, the volatile linear alkanes that are suitable for use in the invention have a vapour pressure ranging from 0.4 to 600 Pa, preferentially from 1 to 200 Pa and even more preferentially from 3 to 60 Pa, at room temperature (25° C.).

According to one embodiment, a volatile linear alkane that is suitable for use in the invention may have a flash point that is within the range from 30 to 120° C. and more particularly from 40 to 100° C. The flash point is in particular measured according to the standard ISO 3679.

According to one embodiment, the volatile linear alkanes that are suitable for use in the invention may be linear alkanes including from 7 to 15 carbon atoms, preferably from 8 to 14 carbon atoms and better still from 9 to 14 carbon atoms.

More preferably, the volatile linear alkanes that are suitable for use in the invention may be linear alkanes including from 10 to 14 carbon atoms and even more preferentially from 11 to 14 carbon atoms.

A volatile linear alkane that is suitable for use in the invention may advantageously be of plant origin.

According to a particular embodiment of the invention, the fatty medium of the composition is oily. More particularly, the composition comprises one or more oils, preferably non-silicone oils, notably hydrocarbon-based oils.

The term “hydrocarbon-based oil” means an oil consisting of carbon and hydrogen atoms.

Preferably, the liquid fatty substances of the invention are chosen from hydrocarbons, fatty alcohols, fatty esters, silicones and fatty ethers, or mixtures thereof. More particularly, the fatty substances of the invention are not (poly)oxyalkylenated.

The term “liquid hydrocarbon” means a hydrocarbon composed solely of carbon and hydrogen atoms, which is liquid at ordinary temperature (25° C.) and at atmospheric pressure (760 mmHg; i.e. 1.013×10⁵ Pa).

More particularly, the liquid hydrocarbons are chosen from:
linear or branched, optionally cyclic, C₆-C₁₆ alkanes. Examples that may be mentioned include hexane, undecane, dodecane, tridecane, and isoparaffins, for instance isohexadecane, isododecane and isodecane;
linear or branched hydrocarbons of mineral, animal or synthetic origin, containing more than 16 carbon atoms, such as liquid paraffins, liquid petroleum jelly, polydecenes hydrogenated polyisobutene such as Parleam®, and squalane.

In a preferred variant, the liquid hydrocarbon(s) are chosen from liquid paraffins and liquid petroleum jelly.

The term “liquid fatty alcohol” means a non-glycerolated and non-oxyalkylenated fatty alcohol that is liquid at ordinary temperature (25° C.) and at atmospheric pressure (760 mmHg; i.e. 1.013×10⁵ Pa).

Preferably, the liquid fatty alcohols of the invention include from 8 to 30 carbon atoms, more preferentially C₁₀-C₂₂, even more preferentially C₁₄-C₂₀, better still C₁₆-C₁₈.

The liquid fatty alcohols of the invention may be saturated or unsaturated.

The saturated liquid fatty alcohols are preferably branched. They may optionally comprise in their structure at least one aromatic or non-aromatic ring. Preferably, they are acyclic.

More particularly, the saturated liquid fatty alcohols of the invention are chosen from octyldodecanol, isostearyl alcohol and 2-hexyldecanol.

According to another variant of the invention, the fatty substance(s) are chosen from liquid unsaturated fatty alcohols. These liquid unsaturated fatty alcohols contain in their structure at least one double or triple bond. Preferably, the fatty alcohols of the invention bear in their structure one or more double bonds. When several double bonds are present, there are preferably two or three of them, and they may be conjugated or non-conjugated.

These unsaturated fatty alcohols may be linear or branched.

They may optionally comprise in their structure at least one aromatic or non-aromatic ring. Preferably, they are acyclic.

More particularly, the liquid unsaturated fatty alcohols of the invention are chosen from oleyl alcohol, linolyl alcohol, linolenyl alcohol and undecylenyl alcohol.

Oleyl alcohol is most particularly preferred.

The term “liquid fatty ester” or “ester oil” means a compound comprising one or more ester groups derived from a fatty acid and/or from a fatty alcohol and that is liquid at ordinary temperature (25° C.) and at atmospheric pressure (760 mmHg; i.e. 1.013×10⁵ Pa).

The esters are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms in the esters being greater than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isodecyl neopentanoate, isostearyl neopentanoate, and C₁₀-C₂₂ and preferably C₁₂-C₂₀ alkyl (iso)stearates such as isopropyl isostearate.

Esters of C₄-C₂₂ dicarboxylic or tricarboxylic acids and of C₁-C₂₂ alcohols and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may notably be made of diethyl sebacate, diisopropyl sebacate, bis(2-ethylhexyl) sebacate, diisopropyl adipate, di-n-propyl adipate, dioctyl adipate, bis(2-ethylhexyl) adipate, diisostearyl adipate, bis(2-ethylhexyl) maleate, triisopropyl citrate, triisocetyl citrate, triisostearyl citrate, glyceryl trilactate, glyceryl trioctanoate, trioctyldodecyl citrate, trioleyl citrate, neopentyl glycol diheptanoate, and diethylene glycol diisononanoate.

The composition may also comprise, as liquid fatty ester, sugar esters and diesters of C₆-C₃₀ and preferably C₁₂-C₂₂ fatty acids. It is recalled that the term “sugar” means oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be notably chosen from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, notably, oleopalmitate, oleostearate and palmitostearate mixed esters.

More particularly, use is made of monoesters and diesters and notably sucrose, glucose or methylglucose monooleate or dioleate, stearate, behenate, oleopalmitate, linoleate, linolenate or oleostearate.

An example that may be mentioned is the product sold under the name Glucate® DO by the company Amerchol, which is a methylglucose dioleate.

Finally, use may also be made of natural or synthetic glycerol esters of mono-, di- or triacids.

Among these, mention may be made of plant oils.

As oils of plant origin or synthetic triglycerides that may be used in the composition of the invention as liquid fatty esters, examples that may be mentioned include:
triglyceride oils of plant or synthetic origin, such as liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil.

Use will preferably be made, as esters according to the invention, of liquid fatty esters derived from monoalcohols.

Isopropyl myristate or isopropyl palmitate is preferred.

The liquid fatty ethers are chosen from liquid dialkyl ethers such as dicaprylyl ether.

According to a preferred embodiment of the invention, the composition comprises one or more hydrocarbon-based oils containing from 8 to 16 carbon atoms.

More particularly, the hydrocarbon-based oil(s) containing from 8 to 16 carbon atoms are chosen from:
branched C₈-C₁₆ alkanes, such as C₈-C₁₆ isoalkanes of petroleum origin (also known as isoparaffins), such as isododecane (also known as 2,2,4,4,6-pentamethylheptane), isodecane, isohexadecane and, for example, the oils sold under the Isopar or Permethyl trade names,
linear C₈-C₁₆ alkanes, for instance n-dodecane (C₁₂) and n-tetradecane (C₁₄) sold by Sasol under the references, respectively, Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155059 from the company Cognis, and mixtures thereof.

The term “ester oil” means an oily compound containing one or more ester groups in its chemical structure.

The ester oil(s) are particularly chosen from:
oils of plant origin, such as triglycerides consisting of fatty acid esters of glycerol in which the fatty acids may have varied chain lengths from C₄ to C₂₄, these chains possibly being linear or branched, and saturated or unsaturated; these oils are notably heptanoic acid or octanoic acid triglycerides. The oils of plant origin may be chosen from wheatgerm oil, sunflower oil, grapeseed oil, sesame seed oil, groundnut oil, corn oil, apricot oil, castor oil, shea oil, avocado oil, olive oil, soybean oil, sweet almond oil, palm oil, rapeseed oil, cottonseed oil, coconut oil, hazelnut oil, walnut oil, rice oil, linseed oil, macadamia oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil, musk rose oil and argan oil; shea butter; or alternatively caprylic/capric acid triglycerides such as those sold by the company Stéarinerie Dubois or those sold under the names Miglyol 810®, 812® and 818® by the company Dynamit Nobel;
monoester oils of formula R⁹—C(O)—OR¹⁰ in which R⁹ represents a linear or branched hydrocarbon-based chain including from 5 to 19 carbon atoms and R¹⁰ represents a linear or branched, notably branched, hydrocarbon-based chain containing from 4 to 20 carbon atoms, on condition that R⁹+R¹⁰≥9 carbon atoms and preferably less than 29 carbon atoms, for instance palmitates, adipates, myristates and benzoates, notably diisopropyl adipate and isopropyl myristate; cetearyl octanoate (purcellin oil), isopropyl myristate, isopropyl palmitate, hexyl laurate, isononyl isononanoate, 2-ethylhexyl palmitate, isostearyl isostearate, 2-hexyldecyl laurate, 2-octyldecyl palmitate, 2-octyldodecyl myristate, 2-ethylhexyl hexanoate, isononyl hexanoate, neopentyl hexanoate, caprylyl heptanoate or octyl octanoate;
esters of lactic acid and of C₁₀-C₂₀ alcohol, such as isostearyl lactate, 2-octyldodecyl lactate, myristyl lactate, C₁₂-C₁₃ alkyl lactate (Cosmacol® ELI from Sasol), cetyl lactate or lauryl lactate;
diesters of malic acid and of C₁₀-C₂₀ alcohol, such as diisostearyl malate, di(C₁₂-C₁₃)alkyl malate (Cosmacol® EMI from Sasol), dibutyloctyl malate, diethylhexyl malate or dioctyldodecyl malate;
esters of pentaerythritol and of C₈-C₂₂ carboxylic acid (in particular tetraesters or diesters), such as pentaerythrityl tetraoctanoate, pentaerythrityl tetraisostearate, pentaerythrityl tetrabehenate, pentaerythrityl tetracaprylate/tetracaprate, pentaerythrityl tetracocoate, pentaerythrityl tetraethylhexanoate, pentaerythrityl tetraisononanoate, pentaerythrityl tetrastearate, pentaerythrityl tetraisostearate, pentaerythrityl tetralaurate, pentaerythrityl tetramyristate, pentaerythrityl tetraoleate or pentaerythrityl distearate;
diesters of the following formula (II) R¹¹—O—C(O)—R¹²—C(O)—O—R¹³, with R¹¹ and R¹³, which may be identical or different, representing a linear or branched, saturated or unsaturated (preferably saturated) C₄ to C₁₂ and preferentially C₅ to C₁₀ alkyl chain, optionally containing at least one saturated or unsaturated, preferably saturated, ring, and R¹² representing a saturated or unsaturated C₁ to C₄, preferably C₂ to C₄, alkylene chain, for instance an alkylene chain derived from succinate (in this case R¹² is a saturated C₂ alkylene chain), maleate (in this case R¹² is an unsaturated C₂ alkylene chain), glutarate (in this case R¹² is a saturated C₃ alkylene chain) or adipate (in this case R¹² is a saturated C₄ alkylene chain); in particular, R¹¹ and R¹³ are chosen from isobutyl, pentyl, neopentyl, hexyl, heptyl, neoheptyl, 2-ethylhexyl, octyl, nonyl and isononyl; mention may be made preferentially of dicaprylyl maleate or bis(2-ethylhexyl) succinate;
diesters of the following formula (III) R¹⁴—C(O)—O—R¹⁵—O—C(O)—R¹⁶, with R¹⁴ and R¹⁶, which may be identical or different, representing a linear or branched, saturated or unsaturated (preferably saturated) C₄ to C₁₂ and preferentially C₅ to C₁₀ alkyl chain and R¹⁵ representing a saturated or unsaturated C₁ to C₄ and preferably C₂ to C₄ alkylene chain. Mention may notably be made of 1,3-propanediol dicaprylate (R¹⁴ as C₇ and R¹⁶ as Ca), sold under the name Dub Zenoat by the company Stéarinierie Dubois, or dipropylene glycol dicaprylate;
the carbonate oils may be chosen from the carbonates of formula R¹⁷—O—C(O)—O—R¹⁸, with R¹⁷ and R¹⁸, which may be identical or different, representing a linear or branched C₄ to C₁₂ and preferentially C₆ to C₁₀ alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 All from BASF), dibutyl carbonate, dineopentyl carbonate, dipentyl carbonate, dineoheptyl carbonate, diheptyl carbonate, diisononyl carbonate or dinonyl carbonate and preferably dioctyl carbonate.

In particular, the fatty substance(s) b) are chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil or cereal germ oil;
linear, branched or cyclic esters containing more than 6 carbon atoms, notably 6 to 30 carbon atoms; and notably isononyl isononanoate; and more particularly esters of formula R—C(O)—O—R′ in which R represents a higher fatty acid residue including from 7 to 19 carbon atoms and R′ represents a hydrocarbon-based chain including from 3 to 20 carbon atoms, such as palmitates, adipates, myristates and benzoates, notably diisopropyl adipate and isopropyl myristate;
hydrocarbons and notably volatile or non-volatile, linear, branched and/or cyclic alkanes, such as C₅-C₆₀ isoparaffins, which are optionally volatile, such as isododecane, Parleam (hydrogenated polyisobutene), isohexadecane, cyclohexane or Isopars; or else liquid paraffins, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof.

Preferably, the composition comprises, in the fatty medium, at least one oil chosen from:
plant oils formed by fatty acid esters of polyols, in particular triglycerides,
esters of formula RC(O)—OR′ in which R represents a higher fatty acid residue including from 7 to 19 carbon atoms and R′ represents a hydrocarbon-based chain including from 3 to 20 carbon atoms,
volatile or non-volatile, linear or branched C₈-C₃₀ alkanes,
volatile or non-volatile, non-aromatic cyclic C₅-C₁₂ alkanes,
ethers containing 7 to 30 carbon atoms,
ketones containing 8 to 30 carbon atoms,
aliphatic fatty monoalcohols containing 12 to 30 carbon atoms, the hydrocarbon-based chain not including any substitution groups, and
mixtures thereof.

Preferably, when the copolymer is such that the alkyl group R¹ comprises from 6 to 9 carbon atoms, the fatty substance(s) b) are chosen from apolar hydrocarbon-based oils containing from 8 to 14 carbon atoms in the absence of monoalcohol containing from 2 to 6 carbon atoms.

Preferably, when the copolymer is such that the alkyl group R¹ comprises 9 carbon atoms, the fatty substance(s) b) are chosen from hydrogenated polyisobutylenes.

In particular the molar percentage of units (A) is greater than the molar percentage of units (B).

In particular, the fatty substance(s) are chosen from non-silicone oils; preferably, the liquid fatty substance(s) are chosen from:
ester oils, carbonate oils; and
branched apolar hydrocarbon-based oils containing from 8 to 14 carbon atoms; as a mixture with
a monoalcohol containing from 2 to 6 carbon atoms preferably in a monoalcohol/branched apolar hydrocarbon-based oil weight ratio ranging from 1/99 to 10/90.

Advantageously, the composition comprises one or more fatty substances, which are notably liquid at 25° C. and at atmospheric pressure, preferably one or more oils, of the fatty medium in a content ranging from 2% to 99.9% by weight, relative to the total weight of the composition, preferably ranging from 5% to 90% by weight, preferably ranging from 10% to 80% by weight, preferably ranging from 20% to 80% by weight.

According to one embodiment of the invention, the composition comprises an aqueous phase. The composition is notably formulated as aqueous lotions or as water-in-oil or oil-in-water emulsions or as multiple emulsions (oil-in-water-in-oil or water-in-oil-in-water triple emulsion (such emulsions are known and described, for example, by C. Fox in “Cosmetics and Toiletries”—November 1986—Vol. 101—pages 101-112)).

The aqueous phase of the composition contains water and in general other water-soluble or water-miscible solvents such as polar and protic solvents as defined below (see additional solvents).

The composition according to the invention preferably has a pH ranging from 3 to 9, depending on the support chosen.

According to a particular embodiment of the invention, the pH of the composition(s) is neutral or even slightly acidic. Preferably, the pH of the composition is between 6 and 7. The pH of these compositions may be adjusted to the desired value by means of acidifying or basifying agents usually used in cosmetics, or alternatively using standard buffer systems.

The term “basifying agent” or “base” means any agent for increasing the pH of the composition in which it is present. The basifying agent is a Brønsted, Lowry or Lewis base. It may be mineral or organic. Particularly, said agent is chosen from a) aqueous ammonia, b) (bi)carbonate, c) alkanolamines such as monoethanolamine, diethanolamine, triethanolamine and derivatives thereof, d) oxyethylenated and/or oxypropylenated ethylenediamines, e) organic amines, f) mineral or organic hydroxides, g) alkali metal silicates such as sodium metasilicates, h) amino acids, preferably basic amino acids such as arginine, lysine, ornithine, citrulline and histidine, and i) the compounds of formula (F) below:

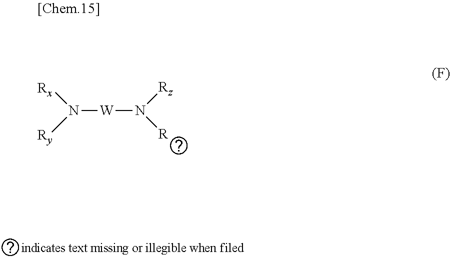

in which formula (F):
W is a divalent C₁-C₆ alkylene radical optionally substituted with one or more hydroxyl groups or a C₁-C₆ alkyl radical, and/or optionally interrupted with one or more heteroatoms such as O or NRᵤ;
Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (E) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Among the mineral or organic hydroxides, mention may be made of those chosen from a) hydroxides of an alkali metal, b) hydroxides of an alkaline-earth metal, for instance sodium hydroxide or potassium hydroxide, c) hydroxides of a transition metal, d) hydroxides of lanthanides or actinides, quaternary ammonium hydroxides and guanidinium hydroxide. The mineral or organic hydroxides a) and b) are preferred.

Among the acidifying agents for the compositions used in the invention, examples that may be mentioned include mineral or organic acids, for instance hydrochloric acid, orthophosphoric acid, sulfuric acid, carboxylic acids, for instance acetic acid, tartaric acid, citric acid or lactic acid, or sulfonic acids.

The basifying agents and the acidifying agents as defined previously preferably represent from 0.001% to 20% by weight relative to the weight of the composition containing them and more particularly from 0.005% to 8% by weight of the composition.

According to a preferred embodiment of the invention, the composition comprises an amount of water of less than or equal to 10% by weight relative to the total weight of the composition. Even more preferentially, the composition comprises an amount of water of less than or equal to 5%, better still less than 2%, even better still less than 0.5%, and is notably free of water. Where appropriate, such small amounts of water may notably be introduced by ingredients of the composition that may contain residual amounts thereof.

Even more preferentially, the composition does not comprise any water.

Advantageously, the composition according to the invention comprises a physiologically acceptable medium. In particular, the composition is a cosmetic composition.

The term “physiologically acceptable medium” means a medium that is compatible with human keratin materials, for instance the skin, the lips, the nails, the eyelashes, the eyebrows or the hair.

The term “cosmetic composition” means a composition that is compatible with keratin materials, which has a pleasant colour, odour and feel and which does not cause any unacceptable discomfort (stinging, tautness or redness) liable to discourage the consumer from using it.

The term “keratin materials” means the skin (body, face, contour of the eyes, scalp), head hair, the eyelashes, the eyebrows, bodily hair, the nails or the lips.

The composition according to the invention may comprise a cosmetic additive chosen from water, fragrances, preserving agents, fillers, colouring agents, UV-screening agents, oils, waxes, surfactants, moisturizers, vitamins, ceramides, antioxidants, free-radical scavengers, polymers and thickeners.

According to a particular embodiment of the invention, the composition comprises a) colouring agents chosen from pigments, direct dyes and mixtures thereof, preferably a) pigments.

The term “pigment” means any pigment of synthetic or natural origin which gives colour to keratin materials. The solubility of the pigments in water at 25° C. and at atmospheric pressure (760 mmHg) is less than 0.05% by weight and preferably less than 0.01%.

They are white or coloured solid particles which are naturally insoluble in the hydrophilic and lipophilic liquid phases usually employed in cosmetics or which are rendered insoluble by formulation in the form of a lake, where appropriate. More particularly, the pigments have little or no solubility in aqueous-alcoholic media.

The pigments that may be used are notably chosen from the organic and/or mineral pigments known in the art, notably those described in Kirk-Othmer's Encyclopedia of Chemical Technology and in Ullmann's Encyclopedia of Industrial Chemistry. Pigments that may notably be mentioned include organic and mineral pigments such as those defined and described in Ullmann's Encyclopedia of Industrial Chemistry “Pigments, organic”, 2005 Wiley-VCH Verlag GmbH & Co. KGaA, Weinheim 10.1002/14356007.a20 371 and ibid, “Pigments, Inorganic, 1. General” 2009 Wiley-VCH Verlag GmbH & Co. KGaA, Weinheim 10.1002/14356007.a20_243.pub3.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopaedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopaedia in the chapter on organic pigments. The organic pigment may notably be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

In particular, the white or coloured organic pigments may be chosen from carmine, carbon black, aniline black, azo yellow, quinacridone, phthalocyanine blue, sorghum red, the blue pigments codified in the Colour Index under the references CI 42090, 69800, 69825, 73000, 74100, 74160, the yellow pigments codified in the Colour Index under the references CI 11680, 11710, 15985, 19140, 20040, 21100, 21108, 47000, 47005, the green pigments codified in the Colour Index under the references CI 61565, 61570, 74260, the orange pigments codified in the Colour Index under the references CI 11725, 15510, 45370, 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in patent FR 2 679 771.

According to a particular embodiment of the invention, the pigment(s) used are pigment pastes of organic pigments such as the products sold by the company Hoechst under the name:
Cosmenyl Yellow IOG: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including:
a mineral core,
at least one binder for fixing the organic pigments to the core, and
at least one organic pigment at least partially covering the core.

The term “lake” refers to dyes adsorbed onto insoluble particles, the assembly thus obtained remaining insoluble during use. The mineral substrates onto which the dyes are adsorbed are, for example, alumina, silica, calcium sodium borosilicate or calcium aluminium borosilicate and aluminium. Among the organic dyes, mention may be made of cochineal carmine.

Examples of lakes that may be mentioned include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053) or D & C Blue 1 (CI 42 090).

The mineral substrates onto which the dyes are adsorbed are, for example, alumina, silica, calcium sodium borosilicate or calcium aluminium borosilicate and aluminium.

Among the dyes, mention may be made of cochineal carmine. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053), D & C Blue 1 (CI 42 090).

An example of a lake that may be mentioned is the product known under the following name: D & C Red 7 (CI 15 850:1).

The pigment(s) may also be pigments with special effects.

The term “pigments with special effects” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye notably of the abovementioned type, and also nacreous pigments based on bismuth oxychloride. They may also be mica particles, at the surface of which are superposed at least two successive layers of metal oxides and/or of organic dyestuffs.

The nacres may more particularly have a yellow, pink, red, bronze, orange, brown, gold and/or coppery colour or tint.

As illustrations of nacres that may be used in the context of the present invention, mention may notably be made of the gold-coloured nacres sold notably by the company BASF under the name Gold 222C (Cloisonne), Sparkle gold (Timica), Gold 4504 (Chromalite) and Monarch gold 233X (Cloisonne); the bronze nacres sold notably by the company Merck under the names Bronze fine (17384) (Colorona) and Bronze (17353) (Colorona), by the company Eckart under the name Prestige Bronze and by the company BASF under the name Super bronze (Cloisonne); the orange nacres sold notably by the company BASF under the names Orange 363C (Cloisonne) and Orange MCR 101 (Cosmica) and by the company Merck under the names Passion orange (Colorona) and Matte orange (17449) (Microna); the brown-tinted nacres sold notably by the company BASF under the names Nu-antique copper 340XB (Cloisonne) and Brown CL4509 (Chromalite); the nacres with a copper tint sold notably by the company BASF under the name Copper 340A (Timica) and by the company Eckart under the name Prestige Copper; the nacres with a red tint sold notably by the company Merck under the name Sienna fine (17386) (Colorona); the nacres with a yellow tint sold notably by the company BASF under the name Yellow (4502) (Chromalite); the red-tinted nacres with a golden tint sold notably by the company BASF under the name Sunstone G012 (Gemtone); the black nacres with a golden tint sold notably by the company BASF under the name Nu-antique bronze 240 AB (Timica); the blue nacres sold notably by the company Merck under the name Matte blue (17433) (Microna), Dark Blue (117324) (Colorona); the white nacres with a silvery tint sold notably by the company Merck under the name Xirona Silver; and the golden-green pinkish-orange nacres sold notably by the company Merck under the name Indian summer (Xirona), and mixtures thereof.

In addition to nacres on a mica support, multilayer pigments based on synthetic substrates such as alumina, silica, sodium calcium borosilicate or calcium aluminium borosilicate, and aluminium, may be envisaged.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the cosmetic composition according to the present invention is generally between 10 nm and 200 μm, preferably between 20 nm and 80 μm and more preferably between 30 nm and 50 μm.

The pigments may be dispersed in the product by means of a dispersant.

The term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. Said agent may be charged: it may be anionic, cationic, zwitterionic or neutral.

According to a particular embodiment of the invention, the dispersants used are chosen from esters of 12-hydroxystearic acid more particularly and of C₈ to C₂₀ fatty acids and of polyols such as glycerol or diglycerol, such as poly(l2-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the cosmetic composition according to the invention may be surface-treated with an organic agent.

Thus, the pigments that have been surface-treated beforehand, which are useful in the context of the invention, are pigments that have totally or partially undergone a surface treatment of chemical, electronic, electrochemical, mechanochemical or mechanical nature, with an organic agent such as those described notably in Cosmetics and Toiletries, February 1990, Vol. 105, pages 53-64, before being dispersed in the composition in accordance with the invention. These organic agents may be chosen, for example, from amino acids; waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; fluorosilicone compounds.

The surface-treated pigments that are useful in the cosmetic composition according to the invention may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in U.S. Pat. No. 4,578,266.

An organic agent covalently bonded to the pigments will preferably be used.

The agent for the surface treatment may represent from 0.1% to 50% by weight, preferably from 0.5% to 30% by weight and even more preferentially from 1% to 10% by weight relative to the total weight of the surface-treated pigments.

Preferably, the surface treatments of the pigments are chosen from the following treatments:
a PEG-silicone treatment, for instance the AQ surface treatment sold by LCW;
a chitosan treatment, for instance the CTS surface treatment sold by LCW;
a triethoxycaprylylsilane treatment, for instance the AS surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a lauroyllysine treatment, for instance the LL surface treatment sold by LCW;
a lauroyllysine dimethicone treatment, for instance the LL/SI surface treatment sold by LCW;
a magnesium myristate treatment, for instance the MM surface treatment sold by LCW;
an aluminium dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a disodium stearoyl glutamate treatment, for instance the NAI surface treatment sold by Miyoshi;
a dimethicone/disodium stearoyl glutamate treatment, for instance the SA/NAI surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
a lauroyllysine/aluminium tristearate treatment, for instance the LL-StAI surface treatment sold by Daito;
an octyltriethylsilane treatment, for instance the OTS surface treatment sold by Daito;
an octyltriethylsilane/perfluoroalkyl phosphate treatment, for instance the FOTS surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
a microcrystalline cellulose and carboxymethylcellulose treatment, for instance the AC surface treatment sold by Daito;
a cellulose treatment, for instance the C2 surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.
The composition in accordance with the present invention may furthermore comprise one or more pigments that are not surface-treated.
According to a particular embodiment of the invention, the pigment(s) are mineral pigments.
According to another particular embodiment of the invention, the pigment(s) are chosen from nacres.

According to a particular embodiment of the invention, the dispersant is present with organic or inorganic pigments in particulate form of submicron size.

The term “submicron” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in an amount (dispersant:pigment) of between 0.5:1 and 2:1, particularly between 0.75:1 and 1.5:1 or better still between 0.8:1 and 1.2:1.

According to a particular embodiment, the dispersant is suitable for dispersing the pigments and is compatible with a condensation-curable formulation.

The term “compatible” means, for example, that said dispersant is miscible in the oily phase of the composition or of the dispersion containing the pigment(s), and it does not retard or reduce the curing. The dispersant is preferably cationic.

The dispersant(s) may therefore have a silicone backbone, such as silicone polyether and dispersants of amino silicone type. Among the suitable dispersants that may be mentioned are:
amino silicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type and are positively charged.

Mention may also be made of dispersants bearing chemical groups that are capable of reacting with the reagents of the oily phase and are thus capable of improving the 3D network formed from the amino silicones. For example, dispersants of epoxy silicone pigments can react chemically with the amino silicone prepolymer amino group(s) to increase the cohesion of the amino silicone film comprising the pigment(s).

Preferably, the pigment(s) are chosen from carbon black, iron oxides, notably black iron oxides, and micas coated with iron oxide, triarylmethane pigments, notably blue and purple triarylmethane pigments, such as Blue 1 Lake, azo pigments, notably red azo pigments, such as D & C Red 7, alkali metal salt of lithol red, such as the calcium salt of lithol red B, even more preferentially red iron oxides.

The colouring agents may be chosen from direct dyes.

The term “direct dye” means natural and/or synthetic dyes, other than oxidation dyes. These are dyes that will spread superficially on the fiber.

They may be ionic or nonionic, preferably cationic or nonionic, i.e. as sole dyes.

These direct dyes are chosen, for example, from neutral, acidic or cationic nitrobenzene direct dyes, neutral, acidic or cationic azo direct dyes, tetraazapentamethine dyes, neutral, acidic or cationic quinone and in particular anthraquinone dyes, azine direct dyes, triarylmethane direct dyes, azomethine direct dyes and natural direct dyes.

Examples of suitable direct dyes that may be mentioned include azo direct dyes; (poly)methine dyes such as cyanines, hemicyanines and styryl dyes; carbonyl dyes; azine dyes; nitro(hetero)aryl dyes; tri(hetero)arylmethane dyes; porphyrin dyes; phthalocyanine dyes, and natural direct dyes, alone or as mixtures.

Preferentially, the direct dye(s) contain at least one quaternized cationic chromophore or at least one chromophore bearing a quaternized or quaternizable cationic group.

According to a particular embodiment of the invention, the direct dyes comprise at least one quaternized cationic chromophore.

As direct dyes according to the invention, mention may be made of the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines such as azacarbocyanines, diazacarbocyanines, diazahemicyanines, hemicyanines, or tetraazacarbocyanines; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; (poly)methines such as dimethines of stilbene or styryl type; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes, or xanthenes.

For the cationic azo dyes, mention may be made particularly of those resulting from the cationic dyes described in Kirk-Othmer's Encyclopedia of Chemical Technology, “Dyes, Azo”, J. Wiley & Sons, updated on Apr. 19, 2010.

Among the azo dyes that may be used according to the invention, mention may be made of the cationic azo dyes described in patent applications WO 95/15144, WO 95/01772 and EP-714954.

According to a preferred embodiment of the invention, the direct dye(s) are chosen from cationic dyes known as “basic dyes”.

Among the azo dyes described in the Colour Index International 3rd edition, mention may be made notably of the following compounds:
Basic Red 22
Basic Red 76
Basic Yellow 57
Basic Brown 16
Basic Brown 17.

Among the cationic quinone dyes, those mentioned in the abovementioned Colour Index International are suitable for use and, among these, mention may be made, inter alia, of the following dyes:
Basic Blue 22
Basic Blue 99.

Among the azine dyes that are suitable for use, mention may be made of those listed in the Colour Index International, for example the following dyes:
Basic Blue 17
Basic Red 2.

Among the cationic triarylmethane dyes that may be used according to the invention, mention may be made, in addition to those listed in the Colour Index, of the following dyes:
Basic Green 1
Basic Violet 3
Basic Violet 14
Basic Blue 7
Basic Blue 26.

Mention may also be made of the cationic dyes described in U.S. Pat. No. 5,888,252, EP 1 133 975, WO 03/029 359, EP 860 636, WO 95/01772, WO 95/15144 and EP 714 954. Mention may also be made of those listed in the encyclopedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons.

Preferably, the cationic direct dyes are chosen from those resulting from dyes of azo and hydrazono type.

According to a particular embodiment, the direct dyes are cationic azo dyes, described in EP 850 636, FR 2 788 433, EP 920 856, WO 99/48465, FR 2 757 385, EP 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4 220 388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1 133 976, GB 738 585, DE 2 527 638, FR 2 275 462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dye(s) comprise(s) a quaternary ammonium group; more preferentially, the cationic charge is endocyclic.

These cationic radicals are, for example, a cationic radical:
bearing an exocyclic (di/tri)(C1-C8)alkylammonium charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic dyes of formulae (III) and (IV) and the azo cationic dyes of formulae (V) and (VI) below:

[Chem. 15]:

Het⁺-C(Rₐ)═N—N(R_{b})—Ar,Q⁻ (III)

Het⁺-N(Rₐ)—N═C(R_{b})—Ar,Q⁻ (IV)

Het⁺-N═N—Ar,Q⁻ (V)

Ar⁺—N═N—Ar″,Q⁻ (VI)

in which formulae (III) to (VI):
Het⁺ represents a cationic heteroaryl radical, preferentially bearing an endocyclic cationic charge, such as imidazolium, indolium or pyridinium, which is optionally substituted, preferentially with at least one (C₁-C₈) alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Rₐ and R_{b}, which may be identical or different, represent a hydrogen atom or a (C₁-C₈)alkyl group, which is optionally substituted, preferentially with a hydroxyl group;
or else the substituent Rₐ with a substituent of Het⁺ and/or R_{b} with a substituent of Ar form, together with the atoms that bear them, a (hetero)cycloalkyl; in particular, Rₐ and R_{b} represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (III) to (VI) as defined previously. More particularly, the cationic direct dyes of formulae (III) to (VI) bearing an endocyclic cationic charge described in patent applications WO 95/15144, WO 95/01772 and EP 714 954. Preferentially the following direct dyes:

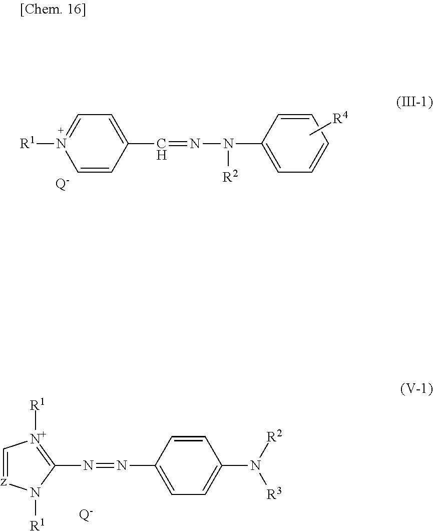

in which formulae (III-1) and (V-1):
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q⁻ is an anionic counterion as defined previously, in particular a halide, such as chloride, or an alkyl sulfate, such as methyl sulfate or mesityl.

In particular, the dyes of formulae (III-1) and (V-1) are chosen from Basic Red 51, Basic Yellow 87 and Basic Orange 31 or derivatives thereof:

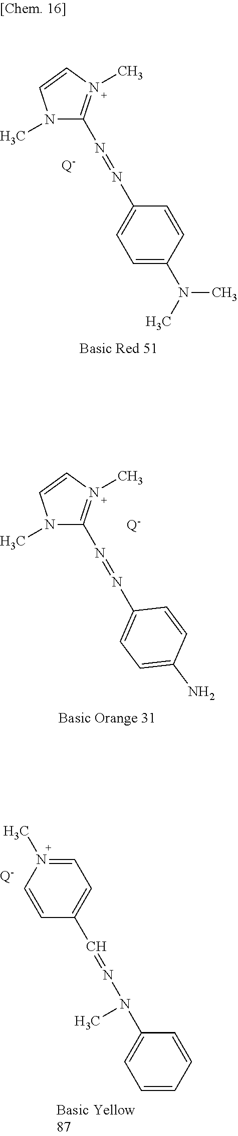

with Q⁻ being an anionic counterion as defined previously, in particular a halide, such as chloride, or an alkyl sulfate, such as methyl sulfate or mesityl.

According to a particular embodiment of the invention, the direct dyes are fluorescent, that is to say that they contain at least one fluorescent chromophore as defined previously.

Fluorescent dyes that may be mentioned include the radicals resulting from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes and xanthenes.

Mention may also be made of the fluorescent dyes described in EP 1 133 975, WO 03/029 359, EP 860 636, WO 95/01772, WO 95/15144 and EP 714 954 and those listed in the encyclopedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to a preferred variant of the invention, the fluorescent dye(s) are cationic and comprise at least one quaternary ammonium radical, such as those of formula (VII) below:

[Chem. 17]:

W⁺—[C(R_{c})═C(R_{d})]ₘ—Ar,Q⁻ (VII)

in which formula (VII):
W⁺ represents a cationic heterocyclic or heteroaryl group, particularly comprising a quaternary ammonium optionally substituted with one or more (C₁-C₈)alkyl groups, optionally substituted notably with one or more hydroxyl groups;
Ar representing an aryl group such as phenyl or naphthyl, optionally substituted preferentially with i) one or more halogen atoms such as chlorine or fluorine; ii) one or more (C₁-C₈)alkyl groups, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
m′ represents an integer ranging from 1 to 4; in particular, m is 1 or 2; more preferentially 1;
R_{c} and R_{d}, which may be identical or different, represent a hydrogen atom or an optionally substituted (C₁-C₈)alkyl group, preferentially of C₁-C₄, or alternatively R_{c} contiguous with W⁺ and/or R_{d} contiguous with Ar form, with the atoms that bear them, a (hetero)cycloalkyl; particularly, R_{c} is contiguous with W⁺ and they form a (hetero)cycloalkyl such as cyclohexyl;
Q⁻ is an organic or mineral anionic counterion as defined previously.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions comprising these natural dyes and notably henna-based poultices or extracts.

According to a particular embodiment of the invention, the amount of colouring agents, notably of pigments, ranges from 0.5% to 40% and preferably from 1% to 20% relative to the weight of the composition and dispersion comprising them.

Advantageously, the composition according to the invention is a makeup composition, in particular a lip makeup composition, a mascara, an eyeliner, an eyeshadow or a foundation.

### Additional Solvents

According to a particular embodiment of the invention, the composition comprises one or more solvents, which are preferably polar and/or protic, other than water in the predominantly fatty medium.

The solvent(s), which are preferably polar and/or protic, other than water are present in the composition in a weight percentage of between 0 and 10% relative to the total weight of the solvent mixture, preferentially between 0.5% and 8%, more particularly between 1% and 5%, such as 2% by weight, relative to the total weight of the composition. Preferably, the solvent(s) are polar protic solvents such as alkanols, more preferentially C₂-C₆ alkanols, such as ethanol.

### The Adjuvants

The composition according to the invention may also comprise one or more fillers, notably in a content ranging from 0.01% to 30% by weight and preferably ranging from 0.01% to 20% by weight relative to the total weight of the composition. The term “fillers” should be understood as meaning colourless or white, mineral or synthetic particles of any shape, which are insoluble in the medium of the composition, irrespective of the temperature at which the composition is manufactured. These fillers notably serve to modify the rheology or texture of the composition.

The composition according to the invention may also contain ingredients commonly used in cosmetics, such as vitamins, thickeners, trace elements, softeners, sequestrants, fragrances, preserving agents, sunscreens, surfactants, antioxidants, agents for combating loss, antidandruff agents and propellants, or mixtures thereof.

The composition according to the invention may be in the form of an anhydrous composition, a water-in-oil emulsion or an oil-in-water emulsion.

The term “anhydrous composition” means a composition containing less than 2% by weight of water, or even less than 0.5% of water, and is notably free of water. Where appropriate, such small amounts of water may notably be introduced by ingredients of the composition that may contain residual amounts thereof.

The invention is illustrated in greater detail in the examples that follow. The amounts are indicated as weight percentages.

### EXAMPLES

The PHAs illustrated in the various examples were prepared in 3-litre chemostats and/or 5-litre Fernbach flasks depending on whether or not a p-oxidation pathway inhibitor is used. The isolation of the PHAs is similar for all the examples obtained.

In a first step, the microorganism generates the PHAs which are stored in intracellular granules, the proportion of which varies as a function of the applied conditions such as the temperature or the nature of the culture medium. The generation of PHA granules may or may not be associated with the growth of the microorganism as a function of the nature of the microorganisms. During the second step, the biomass containing the PHAs is isolated, i.e. separated from the fermentation medium, and then dried. The PHAs are extracted from the biomass before being purified, if necessary.

A mixture of saturated and unsaturated carbon sources is, for certain examples, necessary for the stability of the PHA obtained.

TABLE 2

| Carbon source | CAS |
|---|---|
| Caprylic add (RADIACID 608) | 124-07-2 |
| Nonanoic acid | 112-05-0 |
| Undecylenic acid (10-Undecencic acid) | 112-38-9 |

TABLE 3

| Carbon source | Genus and species | Source |
|---|---|---|
| Caprylic and undecylenoic | Pseudomonas | ATCC ® 47054 ™ |
| acid mixture | putida | |
| Nonanoic and undecylenoic | Pseudomonas | ATCC ® 47054 ™ |
| acid mixture | putida | |

### Example 1: PHA Bearing a Side Chain R¹ Representing a Linear 10% Unsaturated n-octenyl Group and R² Representing an n-Pentyl Group

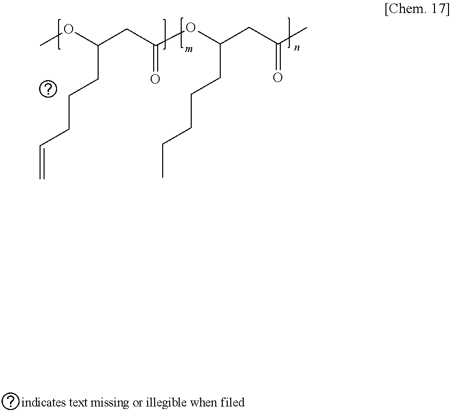

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82, 657-662, 2009.

The microorganism used is Pseudomonas putida KT2440 ATCC® 47054™. The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a nominal dissolved oxygen (O_{D}) value at 30% of saturation. The pH is regulated with 15% aqueous ammonia solution. The temperature of the fermentation medium is regulated at 30° C.

Assembly for the Fed-Batch Growth Fermentation Mode

The fermentation medium is regulated in terms of temperature-pressure of dissolved oxygen and pH (not shown)

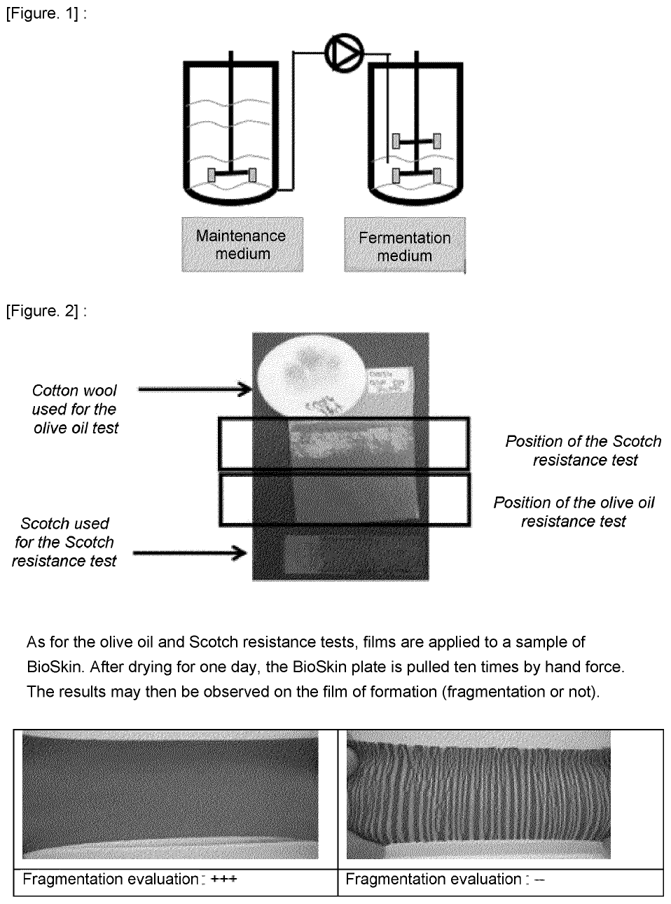

See FIG. 1

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

Composition in Grams Per Litre

TABLE 4

| | CM1 | CM2 | CM3 |
|---|---|---|---|
| Ingredients | « incculum » | « batch » | «maintenance » |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient Broth | 3 | / | |
| Caprylic acid | / | 0.9 | 900 |
| Undecylenic acid | / | 0.1 | 100 |
| Microelement solution | / | 10 | |
| 2N NaOH | | QSP pH = 6.8 | |
| MilliQ water | | QSP 1000 g | |

The composition of the Nutrient Broth, as mass percentages, is 37.5% beef extract and 62.5% peptone. Reference 233000 DIFCO™.

Composition of the Solution of Microelements in Grams Per Litre:

TABLE 5

| Ingredients | Amount | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSo₄•5H₂O | 1.00 | g |

| | |
|---|---|
| MilliQ water (or 0.5N HCL) | QSP 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and are then incubated at 30° C. at 150 rpm for 24 hours. 1.9 L of CM2 “batch” culture medium placed in a presterilized 3 L chemostat are inoculated at OD=0.1 with the 100 mL of preculture. After 4 hours at 30° C. at 850 rpm, the introduction of the maintenance medium is performed by applying the flow rate defined by equation 1.

At the end of the introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, the PHA compound dissolved in the ethyl acetate, is concentrated by evaporation and then dried under high vacuum at 40° C. to constant mass.

The PHA may optionally be purified by successive dissolution and precipitation from an ethyl acetate/ethanol 70% methanol system, for example.

The PHA was fully characterized by spectroscopic and spectrometric methods and is in accordance with the expected chemical structure.

### Example 2: Poly(3-hydroxyoctanoate-co-undecenoate) Containing 10% Unsaturations 100% Grafted with Thiolactic Acid (Compound of Example 1 Grafted with Thiolactic Acid TLA)

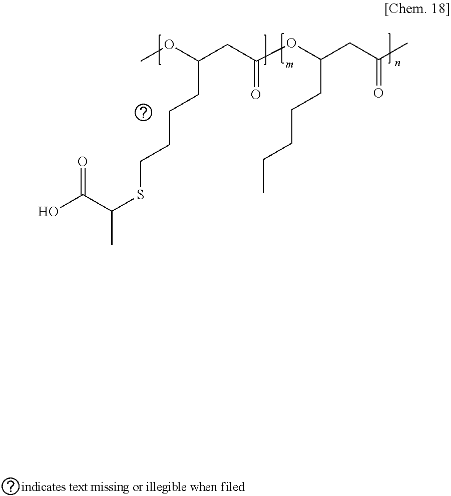

1 g of the compound of Example 1 and 150 mg of thiolactic acid were dissolved in 20 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

20 mL of the reaction medium were then precipitated from a 200 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 2 was fully characterized by spectroscopic and spectrometric methods and is in accordance with the expected chemical structure.

### Example 3: Poly(3-hydroxyoctanoate-co-undecenoate) Containing 10% Unsaturations 100% Grafted with Octanethiol (Compound of Example 1 Grafted with n-octanethiol)

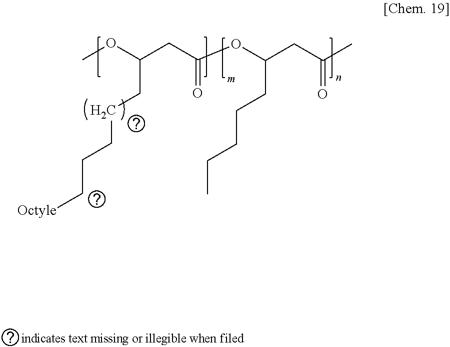

0.5 g of the compound of Example 1 and 125 mg of octanethiol were dissolved in 10 mL of ethyl acetate at room temperature with stirring. 15 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 3 was fully characterized by spectroscopic and spectrometric methods and is in accordance with the expected chemical structure.

### Example 4: Poly(3-hydroxyoctanoate-co-undecenoate) Containing 10% Unsaturations 75% Grafted with 8-mercapto-1-octanol (Compound of Example 1 Grafted with 8-mercapto-1-octanol)

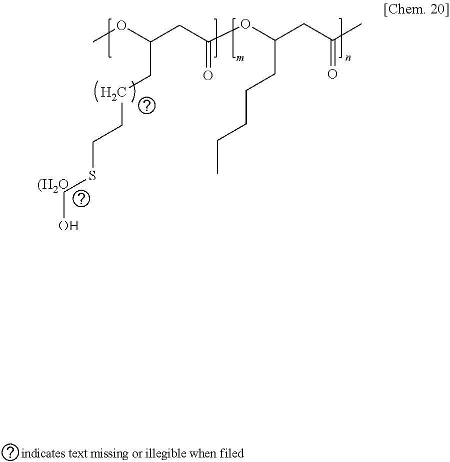

50 mg of the compound of Example 1 and 10 mg of 8-mercapto-1-octanol were dissolved in 5 mL of ethyl acetate at room temperature with stirring. 2 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 4 was fully characterized by spectroscopic and spectrometric methods and is in accordance with the expected chemical structure. Grafting to 75% or 7.5% of functions in total.

### Example 5: Poly(3-hydroxyoctanoate-co-undecenoate) Containing 10% Unsaturations 32% Grafted with Cysteamine (Compound of Example 1 Grafted with Cysteamine)

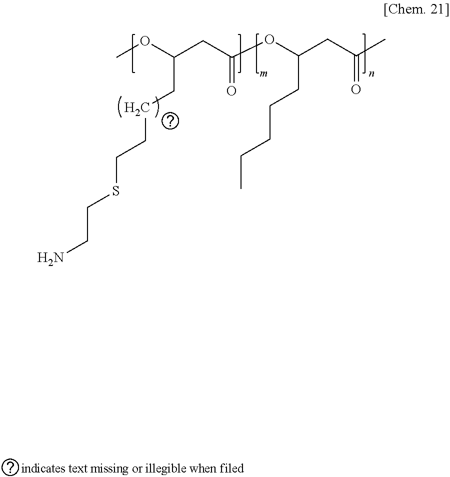

0.5 g of the compound of Example 1 and 54 mg of cysteamine were dissolved in a mixture of 10 mL of dichloromethane and 2 mL of ethanol at room temperature with stirring. 10 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 5 was fully characterized by spectroscopic and spectrometric methods and is in accordance with the expected chemical structure. Grafting to 32% (see the spectrum below) or 3.2% of functions in total.

### Example 6: Poly(3-hydroxyoctanoate-co-undecenoate) Containing 10% Unsaturations 73% Grafted with Cyclohexanethiol (Compound of Example 1 Grafted with CHT)

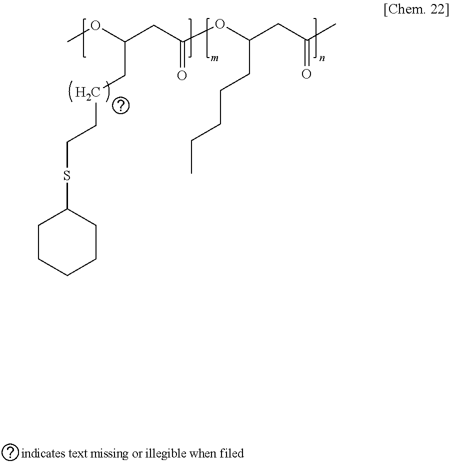

100 mg of the compound of Example 1 and 26 mg of cyclohexanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 6 was fully characterized by spectroscopic and spectrometric methods and is in accordance with the expected chemical structure. Grafting to 73% or 7.3% of functions in total.

### Example 7: Poly(3-hydroxyoctanoate-co-undecenoate) Containing 10% Unsaturations 66% Grafted with 2-furanmethanethiol (FT) (Compound of Example 1 Grafted with FT)

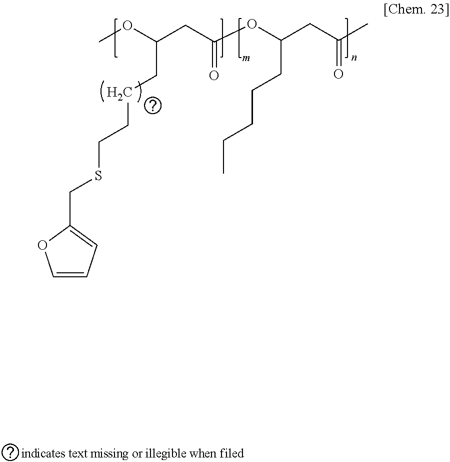

100 mg of the compound of Example 1 and 26 mg of 2-furanmethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 7 was fully characterized by spectroscopic and spectrometric methods and is in accordance with the expected chemical structure. Grafting to 66% or 6.6% of functions in total.

### Example 8: Poly(3-hydroxyoctanoate-co-undecenoate) Containing 10% Unsaturations 66% Grafted with 1-thio-β-D-glucose tetraacetate (Compound of Example 1 Grafted with TGT)

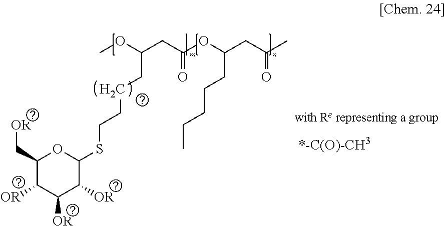

100 mg of the compound of Example 1 and 26 mg of 1-thio-β-D-glucose tetraacetate were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 8 was fully characterized by spectroscopic and spectrometric methods and is in accordance with the expected chemical structure. Grafting to 70% or 7% of functions in total.

### Example 9: Poly(3-hydroxyoctanoate-co-undecenoate) Containing 10% Unsaturations 50% Grafted with 2-phenylethanethiol (PT) (Compound of Example 1 Grafted with PT)

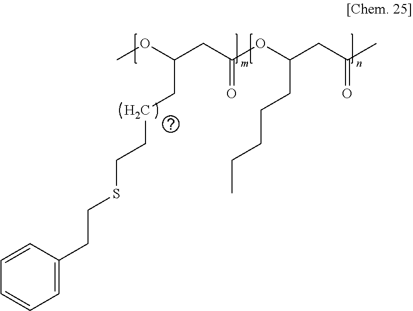

100 mg of the compound of Example 1 and 26 mg of 2-phenylethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 9 was fully characterized by spectroscopic and spectrometric methods and is in accordance with the expected chemical structure. Grafting to 50% or 5% of functions in total.

### Example 10: Poly(3-hydroxyoctanoate-co-undecenoate) Containing 10% Unsaturations 64% Grafted with 4-tert-butylbenzyl mercaptan (TBM) (Compound of Example 1 Grafted with TBM)

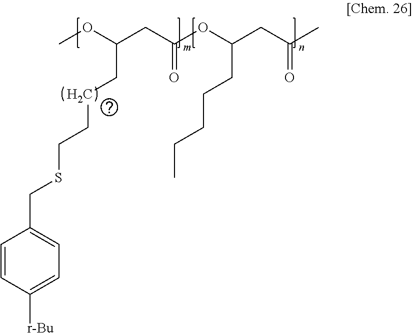

100 mg of the compound of Example 1 and 26 mg of 4-tert-butylbenzyl mercaptan were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 10 was fully characterized by spectroscopic and spectrometric methods and is in accordance with the expected chemical structure. Grafting to 64% or 6.4% of functions in total.

### Example 11: Poly(3-hydroxynonanoate-co-undecenoate) Containing 10% Unsaturations 100% Grafted with Thiolactic Acid

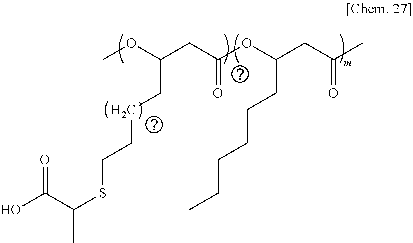

0.1 g of the compound of Example 1 and 15 mg of thiolactic acid were dissolved in 5 mL of chloroform at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 11 was fully characterized by spectroscopic and spectrometric methods and is in accordance with the expected chemical structure. Grafting to 100%.

### Example 12: Poly(3-hydroxynonanoate-co-undecenoate) Containing 5% Unsaturations 100% Grafted with Octanethiol

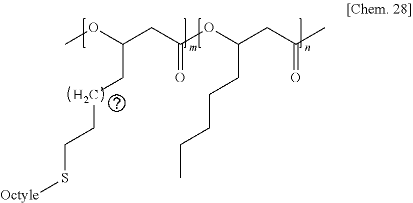

### Preparation of Example 1′: Copolymer of PHA Bearing a Side Chain R¹ Representing an n-hexyl Group and R² Representing an n-hexyl Group

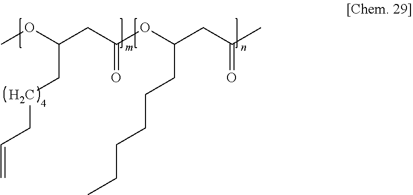

The production process of Example 1 is adapted to that of Example 1′, replacing the n-octanoic acid carbon source of Example 1 with n-nonanoic acid.

The PHA copolymer of Example 1′ was fully characterized by spectroscopic and spectrometric methods and is in accordance with the expected chemical structure, with a degree of unsaturation of 5%.

1 g of the PHA copolymer of Example 1′ and 150 mg of octanethiol were dissolved in 15 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 12 was fully characterized by spectroscopic and spectrometric methods and is in accordance with the expected chemical structure. Grafting to 100%.

### Example 13: Poly(3-hydroxynonanoate-co-undecenoate) Containing 5% Unsaturations 100% Epoxidized

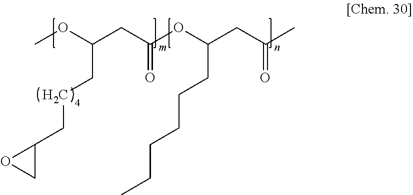

20 g of the PHA copolymer of Example 1′ were dissolved in 80 mL of anhydrous dichloromethane. A suspension of 1.9 g of 77% m-CPBA was prepared with 20 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 13 was fully characterized by spectroscopic and spectrometric methods and is in accordance with the expected chemical structure. Epoxidation to 100%.

### Example 14: Poly(3-hydroxynonanoate-co-undecenoate) Containing 10% Unsaturations 100% Epoxidized

10 g of the PHA copolymer identical to that of Example 1′ but with a degree of unsaturation of 10% were dissolved in 40 mL of anhydrous dichloromethane. A suspension of 1.9 g of 77% m-CPBA was prepared with 10 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 14 was fully characterized by spectroscopic and spectrometric methods and is in accordance with the expected chemical structure. Epoxidation to 100%.

### Example 15: Poly(3-hydroxynonanoate-co-undecenoate) Containing 30% Unsaturations 100% Epoxidized

10 g of the PHA copolymer identical to that of Example 1′ but with a degree of unsaturation of 30% were dissolved in 40 mL of anhydrous dichloromethane. A suspension of 6.2 g of 77% m-CPBA was prepared with 10 mL of anhydrous dichloromethane and added to the mixture with stirring, at room temperature for at least 120 hours.

The reaction medium was then precipitated from a 250 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 15 was fully characterized by spectroscopic and spectrometric methods and is in accordance with the expected chemical structure. Epoxidation to 100%.

### Example 16: Poly(3-hydroxynonanoate-co-undecenoate) Containing 5% Unsaturations 100% Grafted with 4-tert-butylbenzyl Mercaptan (TBM) (Compound of Example 1′ Grafted with TBM)

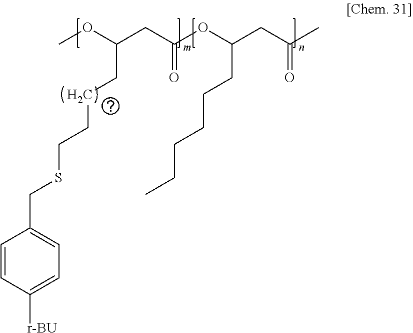

2 g of the PHA copolymer of Example 1′ and 300 mg of 4-tert-butylbenzyl mercaptan were dissolved in 25 mL of ethyl acetate at room temperature with stirring. 25 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 16 was fully characterized by spectroscopic and spectrometric methods and is in accordance with the expected chemical structure. Grafting to 100%.

### Example 17: PHA Bearing a Side Chain R¹ Representing a 5% Linear 8-bromo-n-octanoyl Group and R² Representing a n-hexyl Group

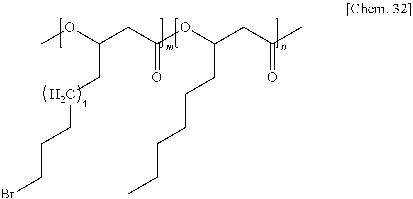

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82, 657-662, 2009.

The microorganism used is Pseudomonas putida KT2440 ATCC® 47054™. The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a dissolved oxygen (O_{D}) value at 30% of saturation. The pH is regulated with a solution composed of ammonia and glucose with a final mass of respectively 15% and 40%. The temperature of the fermentation medium is regulated at 30° C.

Assembly for the Fed-Batch Growth Fermentation Mode:

The fermentation medium is regulated in terms of temperature-pressure of dissolved oxygen and pH (not shown)

See FIG. 1

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium defined CM3 “maintenance” is used for the batch, or maintenance, feeding of the fermentation with the carbon sources of interest at a flow rate calibrated as a function of the growth of the microorganism.

TABLE 6

| | CM1 | CM2 | CM3 |
|---|---|---|---|
| Ingredients in grams per litre | “inoculum” | “batch” | “maintenance” |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient broth | 3 | / | |
| Nonanoic acid | / | 1 | 923 |
| 11-Bromoundecanoic acid | / | 0 | 77 |
| Microelement solution | / | 10 | |

| | |
|---|---|
| 2N NaOH | QSP pH = 6.8 |
| MilliQ water | QSP 1000 g |

The composition of the Nutrient Broth, as mass percentages, is 37.5% beef extract and 62.5% peptone. Reference 233000 DIFCO™.

TABLE 7

| Ingredients in grams per litre | Amount | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or 0.5N HCI) | QSP 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 3 L chemostat are inoculated at OD=0.1 with 100 mL of preculture. After 4 hours at 30° C. at 850 rpm, the introduction of the maintenance medium is performed by applying the flow rate defined by equation 1.

At the end of introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, composed of PHA dissolved in ethyl acetate, is concentrated by evaporation and then dried under high vacuum at 40° C. to constant mass.

The PHA may optionally be purified by successive dissolution and precipitations in an ethyl acetate/ethanol 70% methanol system for instance.

The PHA was fully characterized by spectroscopic and spectrometric methods and is in accordance with the expected chemical structure: 95 molar % of units (B) for which R²=n-hexyl (71%) and n-butyl (24%) and 5 molar % of units (A) for which R¹=8-bromo-n-octanyl (5.9%) and 6-bromo-n-hexyl (0.2%).

### Evaluations

In a first stage, a film is prepared on a contrast card with a film spreader (speed: 50 mm/s-Cylinder: 100 μm). The film is left to dry for 24 hours at room temperature. Once dry, the film has a thickness of about 40 μm.

For the PHA copolymers of Examples 1 to 4 that are soluble in isododecane or an isododecane/ethanol mixture, evaluation of the cosmetic properties on a dry film was performed.

In a first stage, a film is prepared on a contrast card with a film spreader (speed: 50 mm/s-Cylinder: 100 μm). The film is left to dry for 24 hours at room temperature. Once dry, the film has a thickness of about 40 μm.

Three evaluations are performed on the dry film: Resistance to fats, gloss and tackiness

### Measurement of the Resistance to Fats

Three drops of olive oil or sebum or water were deposited on the dry film present on the black part of the contrast card. Each drop corresponds to about 10 μL of olive oil (use of a micropipette).

The drop is left in contact with the dry film for two times: 5 minutes and 30 minutes. Once the time has elapsed, the drop of olive oil or sebum or water is wiped off and observation of the deterioration of the polymer film is performed. If the film was damaged by the drop of olive oil or sebum or water, the polymer film is regarded as being non-resistant to olive oil or to sebum.

Evaluations of the Polymers Only Soluble in Isododecane and Isododecane/Ethanol for Resistance to Water, Oil and Sebum:

TABLE 8

| Tests | Example 12 | Example 13 | Example 16 | Example 17 |
|---|---|---|---|---|
| Resistance to | +++ | +++ | +++ | +++ |
| water | | | | |
| Resistance to | +++ | +++ | +++ | +++ |
| olive oil | | | | |
| Resistance to | ++ | ++ | ++ | ++ |
| sebum | | | | |

It is seen that the PHA copolymers of the invention make it possible to obtain dry, homogeneous films that are particularly resistant to water, olive oil and sebum.

Measurement of the Gloss

Measurement of the gloss with a glossmeter on the black part of the contrast card. The gloss is read at an angle of 20° (the most discerning angle).

Evaluations of the Gloss on the Polymers Alone Soluble in Isododecane and Isododecane/Ethanol:

TABLE 9

| Tests | Example 12 | Example 13 | Example 16 | Example 17 |
|---|---|---|---|---|
| Gloss at | Glossy (50) | Not glossy (3) | Glossy (70) | Not glossy (3) |
| 20° | | | | |

The tack was evaluated in a sensory and qualitative manner by touching the dry film with a finger.

It is seen that Example 13 tested does not have a tacky feel.

### Measure of the Resistance Vs Water/Oil and Adhesive Tape can Also be Evaluated

Mixing of the polymer dissolved in isododecane or isododecane/ethanol with the pigment for 2 minutes at 3500 rpm. The evaluations are performed on BioSkin. In a first stage, a film of each formulation is deposited on a BioSkin sample by means of a film spreader. The thickness of the wet film is 100 μm. The films are dried for 24 hours at room temperature. Once the films are dry, the tests may be performed.

### Resistance to Olive Oil/Sebum

0.5 mL of olive oil or sebum is applied to the film of formulation. After 5 minutes, the olive oil or sebum is removed by wiping 15 times with cotton wool. The deterioration of the film following contact with the olive oil or the sebum is thus examined (see FIG. 2).

### Resistance to Adhesive Tapes

A strip of adhesive tape (of Scotch® type) is applied to the film of formulation. A weight is applied to the strip of said tape for 30 seconds. The adhesive tape is then removed and mounted on a slide holder so as to observe the result. The adherence of the film to the support is thus evaluated (see FIG. 2).

### Example 18: Poly(3-HydroxyNonanoate-co-Undécenoate) with 5% (PHNUn5) and Grafted at 100% with 2-(Trimethylsilyl)ethanthiol (PHNUn5-g-TMS)

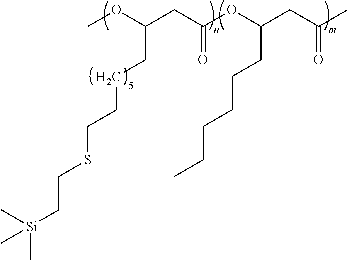

In order to synthesize the intermediate mc1-PHA with linear side chain R¹═C₈ alkenyle group and R² n-hexyl with unsaturated at 5% (PHNUn5) the process is identical than the one discloses in Example 11.

Poly (3-HydroxyNonanoate-co-Undecenoate) is functionalized at 5% unsaturations with 2-(Trimethylsilyl) ethanthiol (PHNUn5 grafted 2-(Trimethylsilyl) ethanthiol) 1 g of (PHNUn5) and 300 mg of 2-(Trimethylsilyl) ethanthiol (TMS) are dissolved in 10 mL of ethyl acetate at room temperature with stirring. Then 20 mg of 2,2-Dimethoxy-2-phenylacetophenone (IRGACURE 651) is added to the mixture. The medium is then irradiated under a 100W UV lamp at 365 nm (reference) and with stirring for at least 10 minutes.

The reaction medium is then precipitated from a 100 ml mixture of 70/30 vv ethanol/water. A viscous white precipitate is obtained. The latter step is repeated if necessary. The product thus obtained is dissolved in a minimum of ethyl acetate, poured onto a Teflon plate, then dried under dynamic vacuum at 40° C., to obtain a homogeneous film.

The PHA grafted with 2-(Trimethylsilyl) ethanthiol is fully characterized by spectroscopic and spectrometric methods and is in accordance with the expected chemical structure, 100% was grafted.

### Example 19: Poly (3-HydroxyNonanoate-co-Undecenoate) with 1% Unsaturations Grafted at 100% with Thiolactic Acid (PHNUn1 Grafted with TLA)

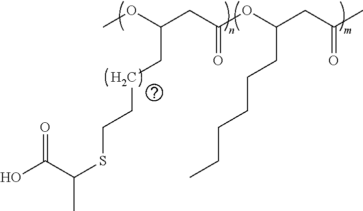

### Preparation of mc1-PHA with Linear Side Chain R¹═C₈ Alkenyl and R²=n-hexyl and Unsaturated at 1% (PHNUn1)

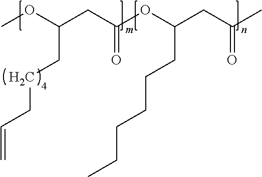

The process for obtaining PHNUn1 is adapted from the article: Applied Microbiololy Biotechnology, Z. Sun, et al., 82, 657-662. (2009).

The microorganism used is Pseudomonas putida KT2440 ATCC® 47054™. The culture mode is carried out under axenic conditions in discontinuous growth fed with a maintenance solution containing a mixture of carbon source at a rate of μ=0.15 h-1 in a 3 L chemostat containing 2.5 L of culture medium. The flow rate of the maintenance feed pump is proportional to the growth of the microorganism according to Equation 1:

$$St = \frac{{X}_{t}}{{Y}_{X / S}} = \frac{{X}_{0}}{{Y}_{X / S}} {e}^{µ . t}$$

The production process is carried out using three separate culture media. The first culture medium defined MC1 “inoculum” is used for the preparation of the preculture. The second culture medium defined MC2 “bach” is used for the non-fed discontinuous growth of the microorganism with the primary carbon sources in the Fernbachs flasks. The third culture medium defined MC3 “maintenance” is used for the batch feeding, or maintenance, of the fermentation with the carbon sources of interest at a rate calibrated according to the growth of the microorganism.

The composition in grams per liter of the three media is described in Table 10:

TABLE 10

| | MC1 | MC2 | MC3 |
|---|---|---|---|
| Ingredients in grams per litre | « inoculum » | « batch » | «maintenance » |
| (NH₄)₂SO₄ | 4.7 | 4.7 | |
| Na₂HPO₄; 7H₂O | 12 | 9 | |
| KH₂PO₄ | 2.7 | 2.03 | |
| MgSO₄; 7H₂O | 0.8 | 1.03 | |
| Nutrient Broth | 3 | / | |
| Nonanoic acid | / | 0.99 | 990 |
| Undecylenic acid | | 0.01 | 10 |
| Microelements Solution | / | 10 | |
| Acrylic acid | / | / | |

| | |
|---|---|
| NaOH 2N | QSP pH = 6.8 |
| MilliQ water | QSP m = 1000 g |

### Composition in Grams Per Liter of Culture Media for Preculture and Maintenance.

The composition of Nutrient Broth in percentage by mass is 37.5% beef extract and 62.5% peptone. Reference 233000 DIFCO™. The composition of the solution of microelements in grams per liter is described in Table 7.

100 mL of preculture is prepared by suspending a cryotube containing 1 mL of the strain with 100 mL “inoculum” culture media at pH adjusted to 6.8 with 2N NaOH in a 250 mL Fernbach flask then incubate at 30° Cat 150 rpm for 24 hours. 1.9 L of “BATCH” MC2 culture medium placed in a previously sterilized 3 L chemostat are inoculated at OD=0.1 with the 100 mL of preculture. After 4 hours at 30° C. at 850 rpm, the introduction of maintenance is performed by applying the flow rate defined by equation 1.

At the end of the introduction, the biomass is isolated by centrifugation and then washed three times with water. The biomass is dried by lyophilization before being extracted with ethyl acetate for 24 hours. The suspension is clarified by filtration through a GF/A filter (Wattman®), the filtrate, composed of PHA dissolved in ethyl acetate, is concentrated by evaporation and then dried under high vacuum at 40° C. to constant mass. The PHA can optionally be purified by solubilization and successive precipitations such as an ethyl acetate/Ethanol 70% methanol mixture.

Then 1 g of (PHNUn1) and 40 mg of thiolactic acid are dissolved in 10 mL of ethyl acetate at room temperature with stirring. 10 mg of 2,2-Dimethoxy-2-Phenylacetophenone (IRGACURE 651) is added to the mixture. The medium is then irradiated under a 100W UV lamp at 365 nm (reference) and with stirring for at least 10 minutes. The reaction medium is then precipitated from a 100 ml mixture of 70/30 vv ethanol/water. A viscous white precipitate was obtained. This step can be repeated. The product thus obtained is dissolved in a minimum of ethyl acetate, poured onto a Teflon plate, then dried under dynamic vacuum at 40° C., to obtain a homogeneous film.

PHA of example 19 grafted with thiolactic acid was characterized spectrometric method and show that the signals characteristic of the unsaturations have completely disappeared. 100% grafting.

### Example 20: Poly (3-HydroxyNonanoate-Co-Undecenoate) at 1% Grafted with Thiolactic Acid Grafted with Dansylcadaverine (PHNUn1-g-TLA-g-Dansylcadaverine)

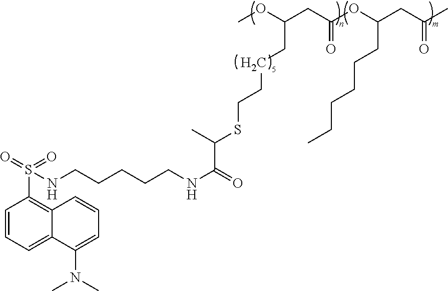

1 g of the preceeding example 19, 12 mg of O-(1H-6-Chlorobenzotriazol-1-yl)-1,1,3,3-tetramethyluronium hexafluorophosphate (HCTU), 10 mg 4-methylmorpholine (NMM) and 12 mg of dansylcadaverine are dissolved in 10 mL of chloroform at room temperature with stirring for 24 h.

The reaction medium is then precipitated from a 100 ml mixture of 70/30 vv ethanol/water. A viscous, slightly yellow colorless precipitate is obtained. This step is repeated if necessary. The product thus obtained is dissolved in a minimum of ethyl acetate, poured onto a Teflon plate, then dried under dynamic vacuum at 40° C., to obtain a homogeneous film.

The PHA is fully characterized by spectroscopic and spectrometric methods and is in accordance with the expected chemical structure.

Evaluations of the Polymers Only Soluble in Isododecane and Isododecane/Ethanol for Resistance to Water, Oil and Sebum:

TABLE 10

| Tests | Example 18 | Example 19 | Example 20 |
|---|---|---|---|
| Resistance to | +++ | +++ | +++ |
| water | | | |
| Resistance to | +++ | +++ | +++ |
| olive oil | | | |
| Resistance to | ++ | ++ | ++ |
| sebum | | | |

It is seen that the PHA copolymers of the invention make it possible to obtain dry, homogeneous films that are particularly resistant to water, olive oil and sebum.

Moreover PHA of the example 20 is evaluated for the resistance vs. water, and sebum under UV lamp. The latter experiment shows an intense and persistent flurescence of the film of example 20.

1. The composition comprising:
a) one or more polyhydroxyalkanoate (PHA) copolymers which contain at least two different repeating polymer units chosen from the units (A) and (B) below, and the optical or geometrical isomers thereof, the organic or mineral acid or base salts thereof, and the solvates thereof:
—[—O—CH(R¹)—CH₂—C(O)—]— unit (A)
—[—O—CH(R²)—CH₂—C(O)—]— unit (B)
in which polymer units (A) and (B):
R¹ represents a hydrocarbon-based chain chosen from i) linear or branched (C₅-C₂₈)alkyl, ii) linear or branched (C₆-C₂₈)alkenyl, iii) linear or branched (C₆-C₂₈)alkynyl;
said hydrocarbon-based chain being:
substituted with one or more atoms or groups chosen from: a) halogen, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, f) cyano, g) iso(thio)cyanate, h) (hetero)aryl, and i) (hetero)cycloalkyl, j) cosmetic active agent chosen from coloured or uncoloured, fluorescent or non-fluorescent chromophores, anti-ageing active agents and fragrances; k) R—X with R representing a group chosen from α) cycloalkyl, β) heterocycloalkyl, γ) (hetero)aryl, δ) cosmetic active agent as defined previously and X representing a′) O, S, N(Rₐ) or Si(R_{b})(R_{c}), b′) S(O)ᵣ, or (thio)carbonyl, c′) or combinations of a′) with b′); r being equal to 1 or 2, Rₐ representing a hydrogen atom, or a (C₁-C₄)alkyl group or an aryl(C₁-C₄)alkyl group; R_{b} and R_{c}, which may be identical or different, represent a (C₁-C₄)alkyl or (C₁-C₄)alkoxy group; and/or
interrupted with one or more heteroatoms a′), b′), c′) or combinations of a′) with b′);
R² represents a cyclic or non-cyclic, linear or branched, saturated or unsaturated hydrocarbon-based group comprising from 3 to 30 carbon atoms; and
b) a fatty medium comprising one or more fatty substances;
it being understood that (A) is different from (B).

2. The composition according to claim 1, in which the PHA copolymer(s) a) contain the repeating unit of formula (I), and the optical or geometrical isomers thereof, the organic or mineral acid or base salts thereof, and the solvates thereof: 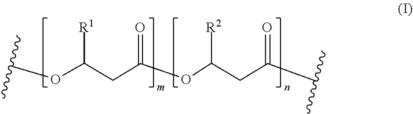
in which formula (I):
m and n are integers greater than or equal to 1.

3. The composition according to claim 1, in which the PHA copolymer(s) a) contain three different repeating polymer units (A), (B) and (C), and preferably consist of three different polymer units (A), (B) and (C), below, and also the optical or geometrical isomers thereof and the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
in which polymer units (A), (B) and (C):
R³ represents a cyclic or non-cyclic, linear or branched, saturated or unsaturated hydrocarbon-based group comprising from 1 to 30 carbon atoms;
optionally substituted with one or more atoms or groups a) to l) and/or optionally interrupted with one or more heteroatoms or groups a′) to c′); and
it being understood that:
(A) is different from (B) and (C), (B) is different from (A) and (C), and (C) is different from (A) and (B); and.

4. The composition according to claim 1, in which the PHA copolymer(s) a) contain four different repeating polymer units (A), (B), (C) and (D), below, and the optical or geometrical isomers thereof, the organic or mineral acid or base salts thereof, and the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
in which polymer units (A), (B), (C) and (D):
R⁴ represents a cyclic or non-cyclic, linear or branched, saturated hydrocarbon-based group comprising from 3 to 30 carbon atoms, optionally substituted with one or more atoms or groups a) to l) and/or optionally interrupted with one or more heteroatoms or groups a′) to c′); and
it being understood that:
(A) is different from (B), (C) and (D), (B) is different from (A), (C) and (D), (C) is different from (A), (B) and (D), and (D) is different from (A), (B) and (C).

5. The composition according to claim 1, in which the PHA copolymer(s) a) contain five different repeating polymer units (A), (B), (C), (D) and (E), below, and the optical or geometrical isomers thereof, the organic or mineral acid or base salts thereof, and the solvates thereof:
—[—O—CH(R¹)—CH₂—C(O)—]— unit (A)
—[—O—CH(R²)—CH₂—C(O)—]— unit (B)
—[—O—CH(R³)—CH₂—C(O)—]— unit (C)
—[—O—CH(R⁴)—CH₂—C(O)—]— unit (D)
—[—O—CH(R⁵)—CH₂—C(O)—]— unit (E)
in which polymer units (A), (B), (C), (D) and (E):
R⁵ represents a cyclic or non-cyclic, linear or branched, saturated hydrocarbon-based group comprising from 3 to 30 carbon atoms, optionally substituted with one or more atoms or groups a) to l) and/or optionally interrupted with one or more heteroatoms or groups a′) to c′);
it being understood that:
(A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D).

6. The composition according to claim 1, in which R¹ represents a linear or branched (C₅-C₂₈)alkyl hydrocarbon-based chain.

7. The composition according to claim 1, in which R¹ represents a hydrocarbon-based chain which is interrupted with one or more atoms or groups chosen from O, S, N(Rₐ), carbonyl, or combinations thereof.

8. The composition according to claim 1, in which R¹ has the following formula —(CH₂)ᵣ—X-(ALK)ᵤ-G, ALK represents a linear or branched (C₁-C₁₀)alkylene chain, r represents an integer inclusively between 6 and 11; u is equal to 0 or 1; and G represents a hydrogen atom or a group chosen from hydroxyl, carboxyl, (di)(C₁-C₄)(alkyl)amino, (hetero)aryl, cycloalkyl, or a sugar.

9. The composition according to claim 1, in which the PHA copolymer(s) a) are such that R² is chosen from linear or branched (C₁-C₂₈)alkyl, and linear or branched (C₂-C₂₈)alkenyl.

10. The composition according to claim 1, in which the PHA copolymer(s) a) are such that the radical R² is a linear or branched (C₁-C₈)alkyl.

11. The composition according to claim 1, in which the PHA copolymer(s) a) are such that:
the unit (A) is present in a molar percentage ranging from 0.1% to 99%; and
the unit (B) is present in a molar percentage ranging from 1% to 40%; and/or
the unit (C) is present in a molar percentage ranging from 0.5% to 20%.

12. (canceled)

13. The composition according to claim 1, in which the fatty medium comprises one or more substances chosen from:
branched C₈-C₁₆ alkanes,
linear C₈-C₁₆ alkanes;
ester oils;
esters of lactic acid and of C₁₀-C₂₀ alcohol;
diesters of malic acid and of C₁₀-C₂₀ alcohol;
esters of pentaerythritol and of C₈-C₂₂ carboxylic acid;
diesters of formula R¹¹—O—C(═O)—R¹²—C(═O)—O—R¹³, with R¹¹ and R¹³, which may be identical or different, representing a linear or branched, saturated or unsaturated C₄ to C₁₂ alkyl chain, optionally containing at least one saturated or unsaturated, preferably saturated, ring, and R¹² representing a saturated or unsaturated C₁ to C₄, alkylene chain;
diesters of formula R¹⁴—C(═O)—O—R¹⁵—O—C(═O)—R¹⁶, with R¹⁴ and R¹⁶, which may be identical or different, representing a linear or branched, saturated or unsaturated C₄ to C₁₂ alkyl chain and R¹⁵ representing a saturated or unsaturated C₁ to C₄ alkylene chain;
the carbonate oils may be chosen from the carbonates of the following formula R¹⁷—O—C(O)—O—R¹⁸, with R¹⁷ and R¹⁸, which may be identical or different, representing a linear or branched C₄ to C₁₂ alkyl chain;
and mixtures thereof.

14. The composition according to claim 1, in which the fatty medium comprises one or more fatty substances in a content ranging from 2% to 99.9% by weight, relative to the total weight of the composition.

15. The composition according to claim 1, in which the fatty medium comprises one or more solvents.

16. The composition according to claim 1, which also comprises one or more colouring agents chosen from pigments, direct dyes and mixtures thereof.

17. A process for treating keratin materials by applying the composition as defined in claim 1.

18. A copolymer PHA which contain at least two different repeating polymer units chosen from the units (A) and (B) below, and the optical or geometrical isomers thereof, the organic or mineral acid or base salts thereof, and the solvates thereof:
—[—O—CH(R¹)—CH₂—C(O)—]— unit (A)
—[—O—CH(R²)—CH₂—C(O)—]— unit (B)
in which polymer units (A) and (B):
R¹ has the following formula —(CH₂)ᵣ—X-(ALK)ᵤ-G, ALK represents a linear or branched-(C₁-C₁₀)alkylene chain, r represents an integer inclusively between 6 and 11 u is equal to 0 or 1; and G represents a hydrogen atom or a group chosen from hydroxyl, carboxyl, (di)(C₁-C₄)(alkyl)amino, (hetero)aryl, and
R² represents a cyclic or non-cyclic, linear or branched, saturated or unsaturated hydrocarbon-based group comprising from 3 to 30 carbon atoms;
R¹ has the following formula —(CH₂)ᵣ—X-(ALK)ᵤ-G, ALK represents a linear or branched (C₁-C₁₀)alkylene chain, r represents an integer inclusively between 6 and 11; u is equal to 0 or 1;
and G represents a hydrogen atom or a group chosen from hydroxyl, carboxyl, (di)(C₁-C₄)(alkyl)amino, (hetero)aryl.

19. A process for preparing the copolymer PHA as defined in claim 18, starting from a PHA copolymer selected from a) to g):
a) a PHA copolymer bearing an unsaturated hydrocarbon-based chain, according to Scheme 1 below: 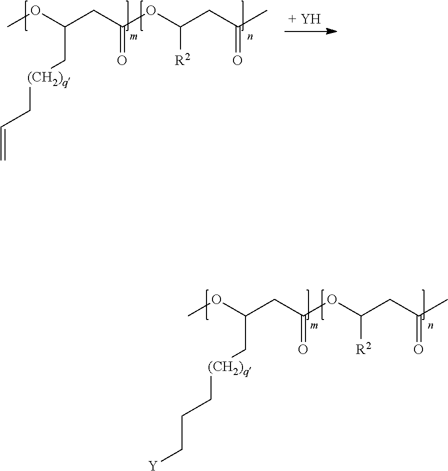
in which Scheme 1:
m and n are integers greater than or equal to 1;
Y represents a group chosen from Hal (halide), hydroxyl, thiol, (di)(C₁-C₄)(alkyl)amino, R—X with R representing a group chosen from α) cycloalkyl β) heterocycloalkyl, γ) (hetero)aryl; δ) a cosmetic active agent as defined previously; ε) (C₁-C₂₀)alkyl, (C₂-C₂₀)alkenyl, (C₂-C₂₀)alkynyl; and X representing a′) O, S, N(Rₐ) or Si(R_{b})(R_{c}) or e) linear or branched (C₁-C₂₀)alkyl, with Rₐ, R_{b} and R_{c} as defined previously;
q′ represents an integer inclusively between 2 and 20;
A) the copolymer PHA with unsaturations may optionally be chemically modified:
via addition reactions;
B) the copolymer PHA with unsaturations may optionally be chemically modified via oxidation reactions, which may or may not be controlled;
b) a PHA copolymer bearing a hydrocarbon-based chain containing an epoxide group, according to Scheme 2 below: 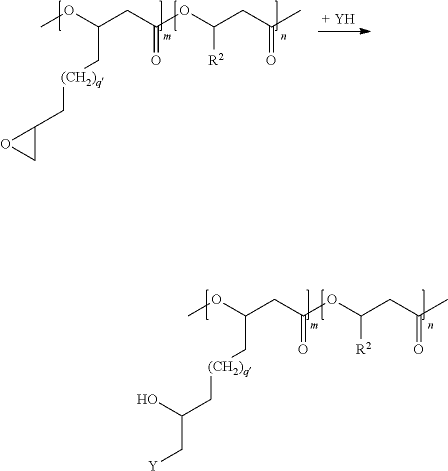
in which Scheme 2, Y, m, n, q′ and R² are as defined in Scheme 1;
c) a PHA copolymer bearing a hydrocarbon-based chain containing a nucleofugal group, according to Scheme 3 below: 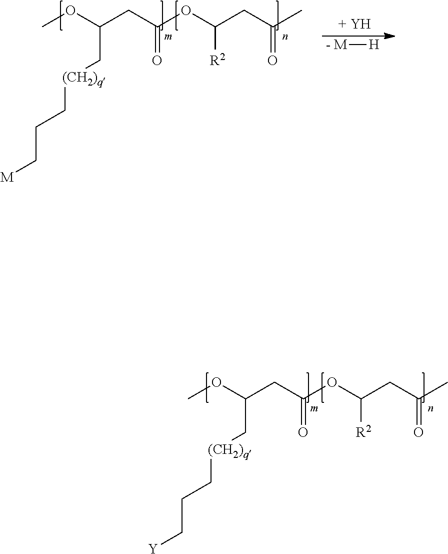
in which Scheme 3 Y, m, n, q′ and R² are as defined in Scheme 1; M corresponds to an organic or inorganic nucleofugal group, which may be substituted with a nucleophilic group;
d) a PHA copolymer bearing a hydrocarbon-based chain containing a cyano group, according to Scheme 4 below: 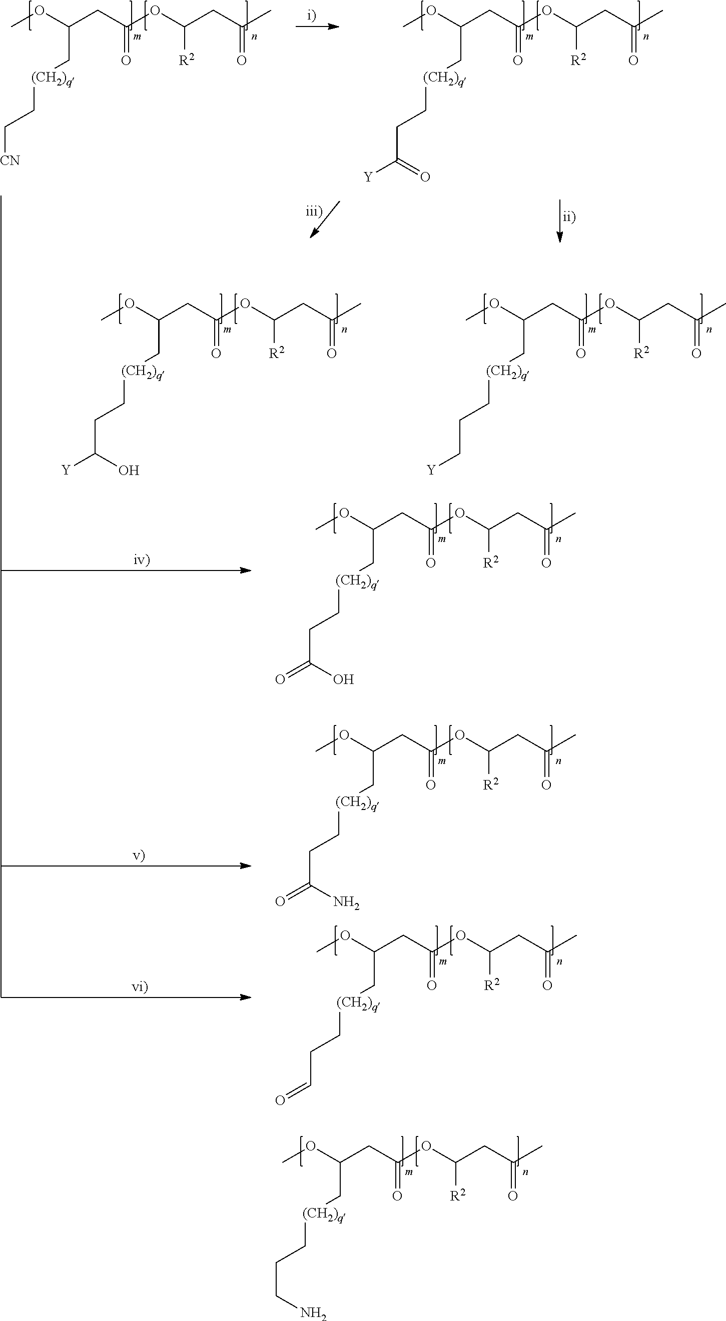
in which Scheme 4 Y, m, n, q′ and R2 are as defined in Scheme 1;
In a first step i), the PHA copolymer bearing a side chain containing a cyano or nitrile group reacts with an organo-alkali metal or organomagnesium compound Y-MgHal, Y—Li or Y—Na, followed by hydrolysis to give the PHA copolymer bearing a side chain containing a group Y grafted with a ketone function; the ketone function may be converted into a thio ketone by thionation; said thio ketone, after total reduction ii) leads to the PHA copolymer bearing a side chain containing a group Y grafted with an alkylene group; alternatively,
said thio ketone may undergo a controlled reduction iii) with a conventional reducing agent to give the PHA copolymer bearing a side chain containing a group Y grafted with a hydroxyalkylene group;
the cyano group of the starting PHA copolymer can react with water after hydration v) to give the amide derivative, or after hydrolysis iv) to the carboxyl derivative;
the cyano group of the starting PHA copolymer may also, after reduction vi), give the amine derivative or the ketone derivative;
e) a PHA copolymer bearing a hydrocarbon-based chain at the chain end, according to Scheme 5 below: 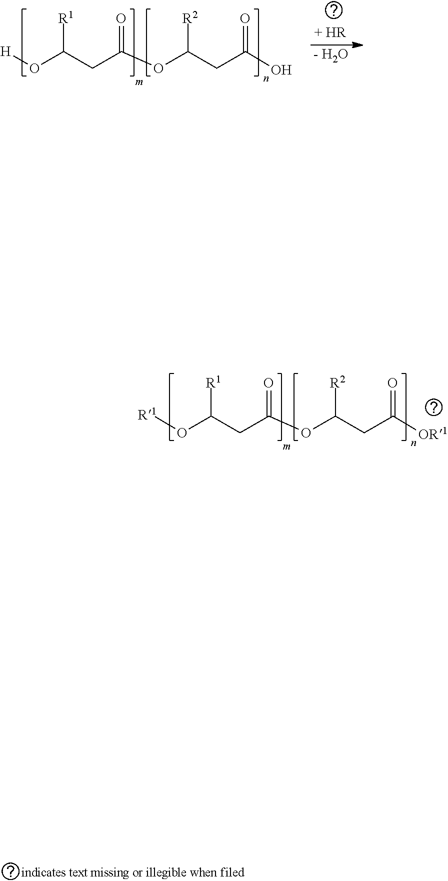
in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; said hydrocarbon-based chain being substituted with one or more atoms or groups chosen from: a) halogens, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl, and i) (hetero)cycloalkyl, j) a cosmetic active agent chosen from coloured or uncoloured, fluorescent or non-fluorescent chromophores, and anti-ageing active agents; or
f) a PHA copolymers with reactive atom or group according to Scheme 6: 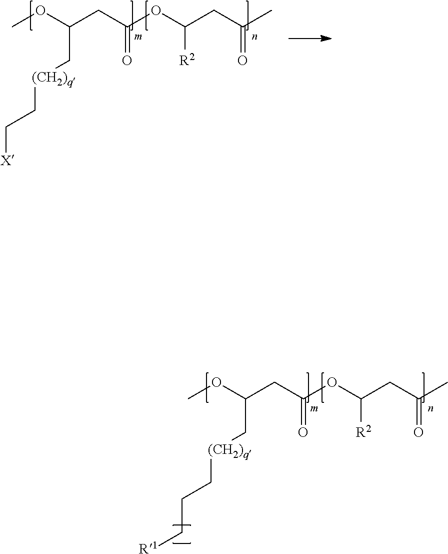
in which Scheme 6 R′¹, R², m, n and Y are as defined previously, and
X′ represents a reactive atom or group that is capable of reacting with an electrophilic E or nucleophilic Nu atom or group to create a Σ covalent bond; if X′ is an electrophilic or nucleofugal group, then it can react with a reagent R′¹—Nu; if X′ is a nucleophilic group Nu, then it can react with R′¹-E to create a Σ covalent bond; the Σ covalent bonds or bonding group that may be generated are listed in the table below, from condensation of electrophiles with nucleophiles:

| Electrophiles [symbol] | Nucleophiles [symbol] u | Covalent bonds |
|---|---|---|
| Activated esters* | Amines | Carboxamides |
| Acyl azides** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salt thereof | Thiols | Thioethers |
| Sulfonic acids and salt thereof | Carboxylic acids | Esters |
| Sulfonic acids and salt thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Azinridines | Thiols | Thioethers |
| Carboxylic acids | Amines | Carboxamides |
| Carboxylic acids | Alcohols | Esters |
| Carbodiimides | Carboxylic acids | N-acylureas |
| Diazolakanes | Carboxylic acids | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*the activated esters of general formula -CO-LG, with LG representing a leaving group such as oxysuccinimidyl, oxybenzotriazolyl or aryloxy, optionally substituted;
**the acyl azides can rearrange to give isocyanate.
g) a PHA functionalized on a side chain, to perform chain-end grafting in a second stage as described in Scheme 7; the reciprocal is also true, in which the chain-end grafting may be performed in a first stage, followed by performing functionalization of a functionalizable side chain in a second stage. 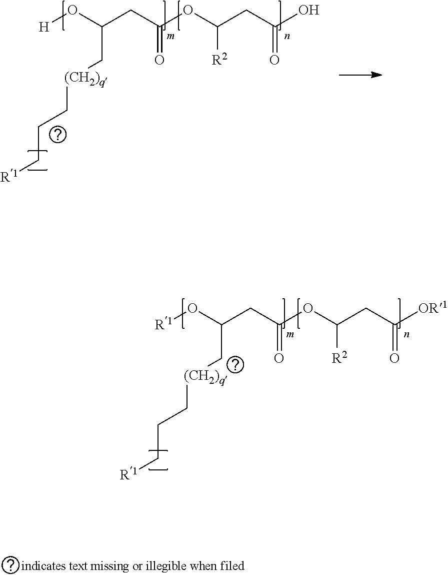
in which Scheme 7 R′¹, R², m, n and Y are as defined previously.

20. A cosmetic process which comprises applying the copolymer PHA as defined in claim 8 to human keratin fibres or human skin.

20. The composition according to claim 1, in which the PHA copolymer(s) a) are such that they comprise the following repeating units, and the optical or geometrical isomers thereof, the organic or mineral acid or base salts thereof, and the solvates thereof:

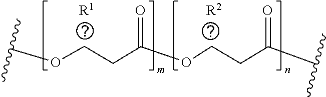

| Compounds | R¹ | R² |
|---|---|---|
| (1) | -(CH₂)₈-S-CH(CH₃)-C(O)-OH | -(CH₂)₄-CH₃ |
| (2) | -(CH₂)₈-S-(CH₂)₇-CH2 | -(CH₂)₄-CH₃ |
| (3) | -(CH₂)₈-S-(CH₂)₈-OH | -(CH₂)₄-CH₃ |
| (4) | -(CH₂)₈-S-(CH2)₂-NH₂ | -(CH₂)₄-CH₃ |
| (5) | -(CH₂)₈-S-Cycl | -(CH₂)₄-CH₃ |
| (6) | -(CH₂)₈-S-CH₂-Fur | -(CH₂)₄-CH₃ |
| (7) | -(CH₂)₈-S-Sug | -(CH₂)₄-CH₃ |
| (8) | -(CH₂)₈-S-(CH₂)₂-Ar | -(CH₂)₄-CH₃ |
| (9) | -(CH₂)₈-S-CH₂-Ar | -(CH₂)₄-CH₃ |
| (10) | -(CH₂)₈-S-CH(CH₃)-C(O)-OH | -(CH₂)₈-CH₃ |
| (11) | -(CH₂)₈-Hal | -(CH₂)₈-CH₃ |
| (12) | -(CH₂)₃-CN | -(CH₂)₈-CH₃ |
| (13) | 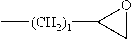 | -(CH₂)₈-CH₃ |
| (14) | -(CH₂)₂-Ar | -(CH₂)₈-CH₃ |

m and n are integers greater than or equal to 1, Hal represents a halogen atom and t represents an integer between 1 and 10,
Ar: represents a (hetero)aryl group;
Ar′: represents a (C₁-C₄)alkyl(hetero)aryl group;
Cycl: represents a cyclohexyl group;
Fur: represents a furyl group;
Sug: represents a sugar group.
